(19) United States
(12) Patent Application Publication
(10) Pub. No.: US 2024/0268830 A1
(43) Pub. Date: Aug. 15, 2024

(54) MULTI-PART MEDICAL DEVICES WITH LOCKING MECHANISM

(71) Applicants: BOSTON SCIENTIFIC SCIMED, INC., MAPLE GROVE, MN (US); Boston Scientific Medical Device Limited, Galway (IE)

(72) Inventors: Ryan Robert Davis, Plymouth, MN (US); Jeffery D. Arnett, Gilbert, AZ (US); Peeyush Tomar, Muzaffarnagar (IN); Neeraj Kumar Sharma, New Delhi (IN)

(73) Assignees: BOSTON SCIENTIFIC SCIMED, INC., MAPLE GROVE, MN (US); Boston Scientific Medical Device Limited, Galway (IE)

(21) Appl. No.: 18/439,047

(22) Filed: Feb. 12, 2024

Related U.S. Application Data
(60) Provisional application No. 63/445,090, filed on Feb. 13, 2023.

(51) Int. Cl. A61B 17/12; A61B 17/00

(52) U.S. Cl. CPC A61B 17/12031; A61B 17/00234; A61B 2017/00243; A61B 2017/00305

(57) ABSTRACT

A variety of medical procedures utilize two or more medical device components, including medical device components where a first medical device component is advanced within a second medical component. An example would be an elongate dilator used in combination with a guide catheter. The guide catheter and the elongate dilator may be adapted to provide a rotational lock mechanism that allows the guide catheter and the elongate dilator to lock together such that rotating one of the components causes rotation of both components. In some instances, the guide catheter and the elongate dilator may cooperate to also provide a relative axial lock between the two components.

### CROSS REFERENCE TO RELATED APPLICATIONS

The present application claims the benefit of and priority to U.S. Provisional Patent Application No. 63/445,090, filed Feb. 13, 2023, the disclosure of which is incorporated herein by reference.

### TECHNICAL FIELD

The disclosure relates generally to medical devices and more particularly to medical devices having a rotational locking mechanism that releasably provides a lock between components.

### BACKGROUND

A variety of medical procedures utilize two or more medical device components, including medical device components where a first medical device component is advanced within a second medical component. An example would be an elongate dilator used in combination with a guide catheter. In some instances, there may be a desire to be able to control the movement of both the first medical device component and the second medical device component without having to physically touch both components simultaneously. In some instances, for example, there may be a desire to provide a rotational lock between the two components such that when a physician or other professional rotates one component, the other components rotates as well. There is an ongoing need for improved medical devices and medical device systems.

### SUMMARY

This disclosure provides design, material, manufacturing method, and use alternatives for medical devices. An example may be found in a medical device assembly. The medical device assembly includes a first elongate medical device having a first elongate shaft having a first proximal region and a first distal region and a first proximal hub secured to the first proximal region. The medical device assembly includes a second elongate medical device having a second elongate shaft having a second proximal region and a second distal region and a second proximal hub secured to the second proximal region. The first elongate medical device is adapted to form a releasable axial and/or rotational lock with the second elongate medical device.

Alternatively or additionally, the second elongate shaft may be adapted to fit within a lumen extending through the first elongate shaft.

Alternatively or additionally, the second proximal hub may be adapted to releasably couple to the first proximal hub when the second elongate shaft is disposed within the lumen extending through the first elongate shaft.

Alternatively or additionally, the second proximal hub may be adapted to form a releasable rotational lock with the first proximal hub.

Alternatively or additionally, the second proximal hub may be adapted to form a releasable axial lock with the first proximal hub.

Alternatively or additionally, the first elongate medical device may be adapted to be advanced over a guidewire in order to reach an atrial septum.

Alternatively or additionally, the second elongate medical device may be adapted to be advanced over the guidewire in combination with the first elongate medical device in order to create or enlarge an aperture formed in the atrial septum.

Alternatively or additionally, the first elongate medical device may include a guide catheter.

Alternatively or additionally, the second elongate medical device may include an elongate dilator.

Alternatively or additionally, the second proximal hub may include a graspable profile.

Another example may be found in a medical device assembly for accessing a left atrial appendage. The medical device assembly includes a guide catheter having a guide catheter shaft and a guide catheter hub secured to a proximal region of the guide catheter shaft. The medical device assembly includes an elongate dilator having an elongate dilator shaft including a distal region adapted to create and/or enlarge an aperture in tissue and a dilator hub secured to a proximal region of the elongate dilator shaft. The guide catheter and the elongate dilator are adapted to form a releasable lock therebetween.

Alternatively or additionally, the elongate dilator shaft may be adapted to fit within a lumen extending through the guide catheter shaft.

Alternatively or additionally, the dilator hub may be adapted to releasably couple to the guide catheter hub when the elongate dilator shaft is disposed within the lumen extending through the guide catheter shaft.

Alternatively or additionally, the dilator hub may be adapted to form a releasable rotational lock with the guide catheter hub.

Alternatively or additionally, the dilator hub may be adapted to form a releasable axial lock with the guide catheter hub.

Alternatively or additionally, the guide catheter may be adapted to be advanced over a guidewire in order to reach an atrial septum.

Alternatively or additionally, the elongate dilator may be adapted to be advanced over the guidewire in combination with the guide catheter in order to create or enlarge an aperture formed in the atrial septum.

Alternatively or additionally, the second proximal hub may include a graspable profile.

Another example may be found in a medical device assembly for implanting a left atrial appendage closure (LAAC) device. The medical device assembly includes a guide catheter having a guide catheter shaft and a guide catheter hub secured to a proximal region of the guide catheter shaft. The medical device assembly includes an elongate dilator having an elongate dilator shaft including a distal region adapted to create and/or enlarge an aperture in tissue and a dilator hub secured to a proximal region of the elongate dilator shaft. The guide catheter and the elongate dilator are adapted to form a releasable lock therebetween.

Alternatively or additionally, the medical device assembly may further include an LAAC device delivery catheter that is adapted to be advanced through the guide catheter once the elongate dilator has been removed.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 provides an illustrative example of part of a procedure for implanting an LAAC (left atrial appendage closure) device, which includes a guide catheter used in combination with an elongate dilator;

FIG. 2A is a side view of an illustrative assembly including a guide catheter and an elongate dilator, with the elongate dilator inserted within the guide catheter but not coupled with the guide catheter;

FIG. 2B is a side view of the illustrative assembly of FIG. 2A with the elongate dilator temporarily coupled with the guide catheter;

FIGS. 3 through 11B are views of an Example A that provides a rotational lock between a guide catheter and an elongate dilator;

FIGS. 12 through 20 are views of an Example B that provides a rotational lock between a guide catheter and an elongate dilator;

FIGS. 21 through 26B are views of an Example C that provides a rotational lock and an axial lock between a guide catheter and an elongate dilator;

FIGS. 27A through 32B are views of an Example D that provides a rotational lock between a guide catheter and an elongate dilator; and

FIGS. 33 through 36B are views of an Example E that provides a rotational lock between a guide catheter and an elongate dilator.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A variety of medical procedures may include the use of two different medical devices that are used in combination. As an example, a first medical device may be used in combination with a second medical device in order to gain access to a particular treatment site and then to carry out an appropriate treatment at the particular treatment site. In some cases, a guidewire may be advanced through the vasculature to reach a particular treatment site. A first medical device may be advanced over the guidewire, and then a second medical device may be advanced through the first medical device. In some instances, there may be a desire to be able to advance and control the first medical device and the second medical device as an assembly, meaning that both the first medical device and the second medical device may be moved, advanced or withdrawn axially without the user being required to separately hold both the first medical device and the second medical device at the same time. In some instances, the first medical device and the second medical device may be rotated as an assembly, meaning that the first medical device and the second medical device may be rotated together without the user being required to separately hold both the first medical device and the second medical device at the same time. In some instances, there can be advantages to being able to advance or rotate the first medical device and the second medical device together, particularly when the first medical device and the second medical device have curved distal regions and/or are steerable.

An illustrative but non-limiting example of a medical procedure that may utilize a first medical device and a second medical device in combination may include reaching the left atrium in order to implant a left atrial appendage closure (LAAC) device. In particular, a first medical device such as a guide catheter may be used in combination with a second medical device such as an elongate dilator. It will be appreciated that two such medical devices may be used in combination in performing any of a variety of different medical procedures. Gaining access to the left atrium in order to deliver and implant an LAAC is merely an example of using a first medical device in combination with a second medical device.

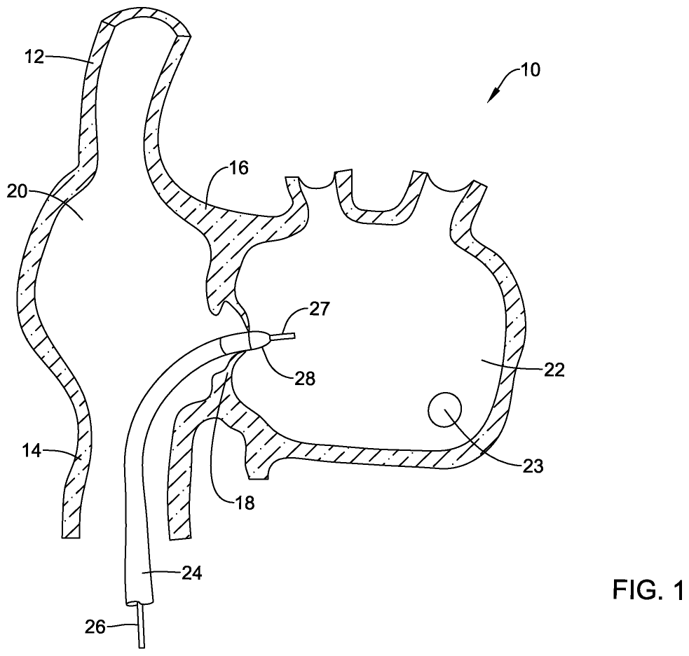

FIG. 1 provides a schematic view of a portion of a person's heart 10, including a superior vena cava 12, an inferior vena cava 14, a septum 16, an atrial septum 18, a right atrium 20 and a left atrium 22. In some cases, the left atrium 22 may include a left atrial appendage (LAA) 23. A composite medical device including a guide catheter 24 may be advanced over a guidewire 26. In some cases, the guidewire 26 may be an RF guidewire that is adapted to utilize RF (radio frequency) energy to cauterize, using a cautery tip 27. In some cases, for example, an RF guidewire may be used to form a small aperture in or near the atrial septum 18. In some cases, a guidewire may have a sharp distal end that may be used to form a puncture.

The puncture through the atrial septum 18 can be done from a position within the right atrium 20. By forming an aperture through the atrial septum 18, it is possible to reach the left atrium 22 from the relative safety of the right side of the heart. In some cases, an elongate medical device including an elongate dilator 28 may be advanced over the guidewire 26, and within the guide catheter 24. Once an aperture has been formed within the atrial septum, the elongate dilator 28 may be advanced over the guidewire and through the aperture in order to widen the aperture. From there, the elongate dilator 28 and guidewire 26 may be removed in order to allow a delivery device carrying an LAAC (left atrial appendage closure) device to be advanced through the guide catheter 24. A variety of devices may be advanced through the guide catheter 24 in order to reach the LAA 23. An illustrative example of a suitable LAAC device includes the Watchman FLX™ LAAC device commercially available from Boston Scientific Corporation.

In some cases, an assembly including the guide catheter 24 and the elongate dilator 28 may be advanced through the inferior vena cava 14 in order to reach the right atrium 20. It will be appreciated that this can represent a tortuous path through the vasculature. In some cases, the guide catheter 24 and/or the elongate dilator 28 may be adapted to have a curved distal end in order to facilitate steering. It will be appreciated that being able to hold the guide catheter 24 and the elongate dilator 28 from relative rotation therebetween may be beneficial in steering the devices through the anatomy. Described herein are several examples of mechanical rotational locking mechanisms that may be used to releasably provide a rotational lock between the guide catheter 24 and the elongate dilator 28. These devices may also provide an axial lock between the guide catheter 24 and the elongate dilator 28.

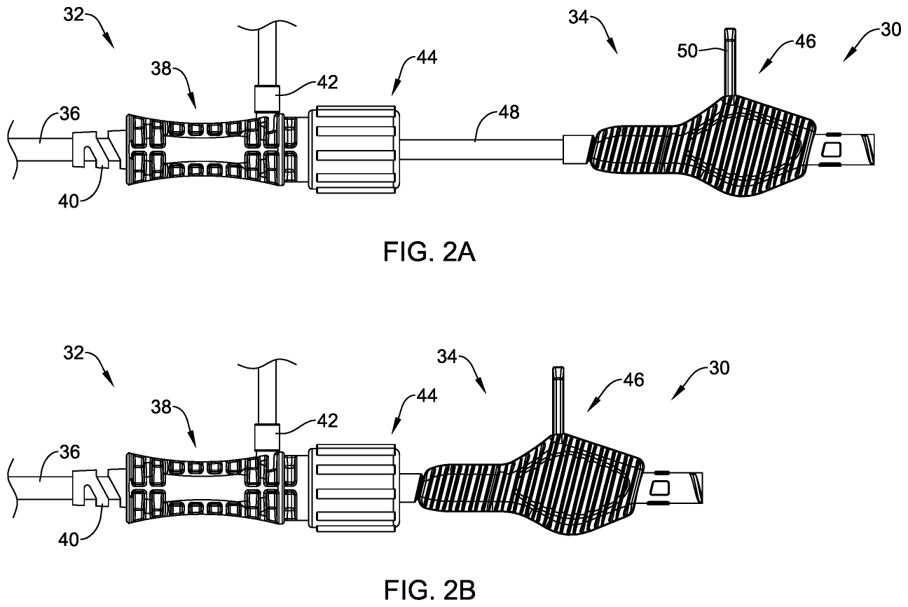

FIG. 2A is a side view of an illustrative assembly 30 that includes a guide catheter 32 and an elongate dilator 34. The guide catheter 32 may be considered as being an example of the guide catheter 24 and the elongate dilator 34 may be considered as being an example of the elongate dilator 28. As will be discussed, the guide catheter 32 and the elongate dilator 34 may be adapted such that the guide catheter 32 and the elongate dilator 34 may be coupled together to provide a releasable rotational lock between the guide catheter 32 and the elongate dilator 34.

The guide catheter 32 includes an elongate shaft 36 that terminates at a proximal end thereof in a proximal hub 38. In some instances, a strain relief 40 extends distally over the elongate shaft 36 a short distance from the proximal hub 38. In some cases, the proximal hub 38 may include one or more fittings such as Luer fittings that allow introduction of various fluids, for example. As shown, the proximal hub 38 includes a fitting 42 disposed on an upper portion of the proximal hub 38. In some cases, as will be discussed, the location of the fitting 42 relative to the proximal hub 38 provides an indication of a direction of curvature of a distal region (not shown) of the elongate shaft 36. In some instances, the distal region of the elongate shaft 36 may curve in an upward direction when the proximal hub 38 (and hence the elongate shaft 36) is oriented with the fitting 42 pointing upwards (as shown). A threaded nut 44 is threadedly engaged with the proximal hub 38. In some instances, the threaded nut 44 may be manipulated by rotating the threaded nut 44 in either a clockwise direction or a counter-clockwise direction in order to open or close a valve that is disposed within the proximal hub 38.

The elongate dilator 34 includes a dilator hub 46 that is adapted to function as a handle. It will be appreciated that the dilator hub 46 is adapted to be easily grasped between the thumb and forefinger of a user, for example. The elongate dilator 34 includes a shaft 48 that extends proximally from the dilator hub 46. While not shown, a distal end of the shaft 48 may be pointed or otherwise be adapted to puncture tissue. In some cases, the distal end of the shaft 48 may be adapted to create an aperture in tissue without any initial aperture made by a guidewire, for example. In some cases, the distal end of the shaft 48 may be adapted to enlarge an existing aperture.

The dilator hub 46 may be adapted to be releasably secured to the proximal hub 38 of the guide catheter 32. By doing so, this provides a connection between the guide catheter 32 and the elongate dilator 34. In some instances, this connection may provide a releasable rotational lock between the guide catheter 32 and the elongate dilator 34. In some instances, the dilator hub 46 may include an indicator 50 that extends away from the dilator hub 46. In some cases, particularly when the shaft 48 of the dilator 34 includes a curved distal region, the orientation of the indicator 50 relative to the dilator hub 46 provides an indication of a direction of curvature of the curved distal region. In some cases, aligning the indicator 50 on the dilator hub 46 with the fitting 42 on the proximal hub 38 provides a corresponding directional alignment between a curved distal region of the elongate shaft 36 and a curved distal region of the shaft 48.

In FIG. 2A, the shaft 48 of the elongate dilator 34 has been inserted into a lumen extending through the proximal hub 38 and the elongate shaft 36 of the guide catheter 32 but the dilator hub 46 has not yet made contact with and been secured relative to the proximal hub 38. In FIG. 2B, the dilator hub 46 is shown secured relative to the proximal hub 38. In some instances, the securement between the proximal hub 38 and the dilator hub 46 may provide a releasable rotation lock between the guide catheter 32 and the elongate dilator 34 such that the guide catheter 32 and the elongate dilator 34 may be rotated together using only a single hand, i.e., the user will only have to hold onto one of the proximal hub 38 and the dilator hub 46. In some instances, this can provide benefits in steering the assembly 30 through the vasculature, particularly in circumstances in which the guide catheter 32 and the elongate dilator 34 both include a distal curvature. This may be particularly useful when the user grasps only the dilator hub 46, as the dilator hub 46 is adapted to be easily grasped.

In some instances, the securement between the proximal hub 38 and the dilator hub 46 may provide a releasable axial lock between the guide catheter 32 and the elongate dilator 34 such that the guide catheter 32 and the elongate dilator 34 may be advanced or withdrawn axially together using only a single hand, i.e., the user will only have to hold onto one of the proximal hub 38 and the dilator hub 46. This may be particularly useful when the user grasps only the dilator hub 46, as the dilator hub 46 is adapted to be easily grasped.

In some instances, the securement between the proximal hub 38 and the dilator hub 46 may provide a releasable axial lock and a releasable rotation lock between the guide catheter 32 and the elongate dilator 34 such that the guide catheter 32 and the elongate dilator 34 may be advanced or withdrawn axially together using only a single hand, i.e., the user will only have to hold onto one of the proximal hub 38 and the dilator hub 46. The user is able to both advance and rotate both the guide catheter 32 and the elongate dilator 34 with a single hand. This may be particularly useful when the user grasps only the dilator hub 46, as the dilator hub 46 is adapted to be easily grasped.

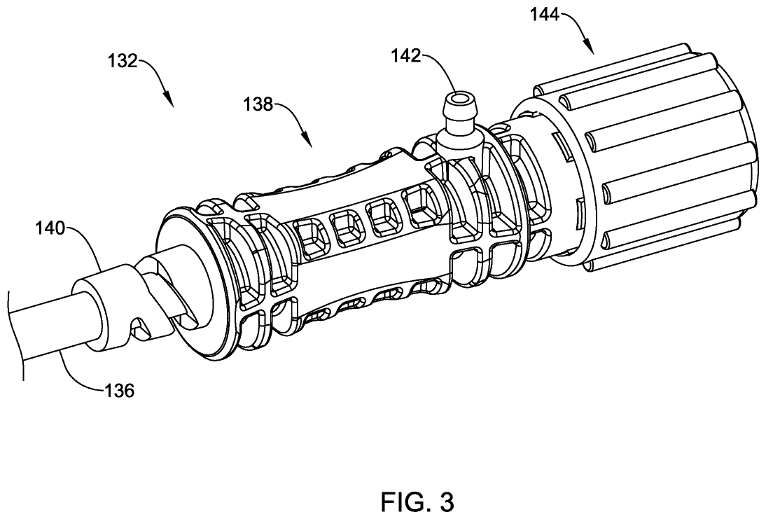

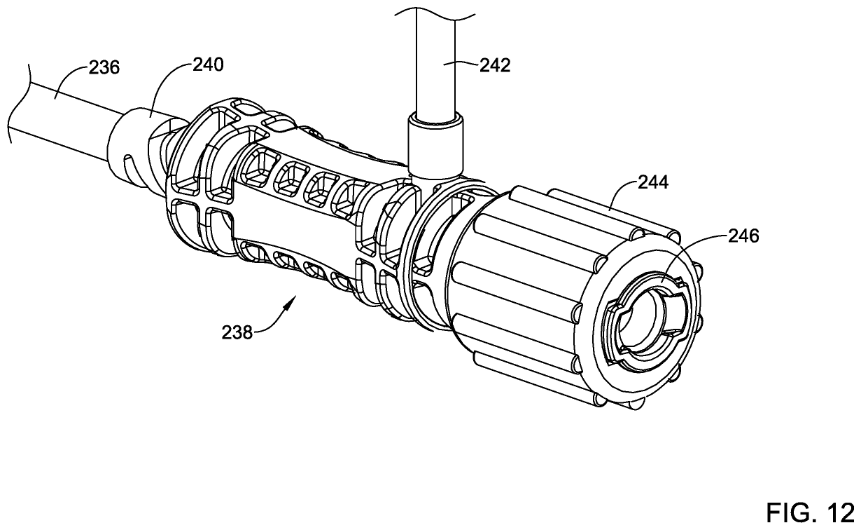

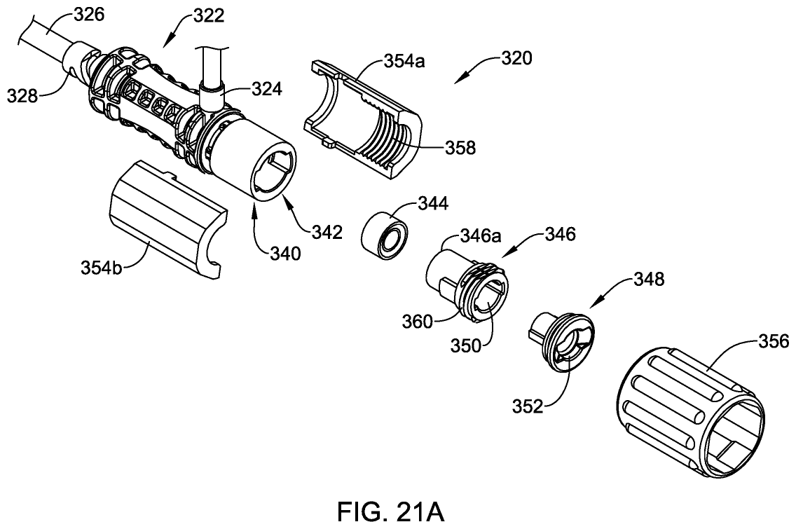

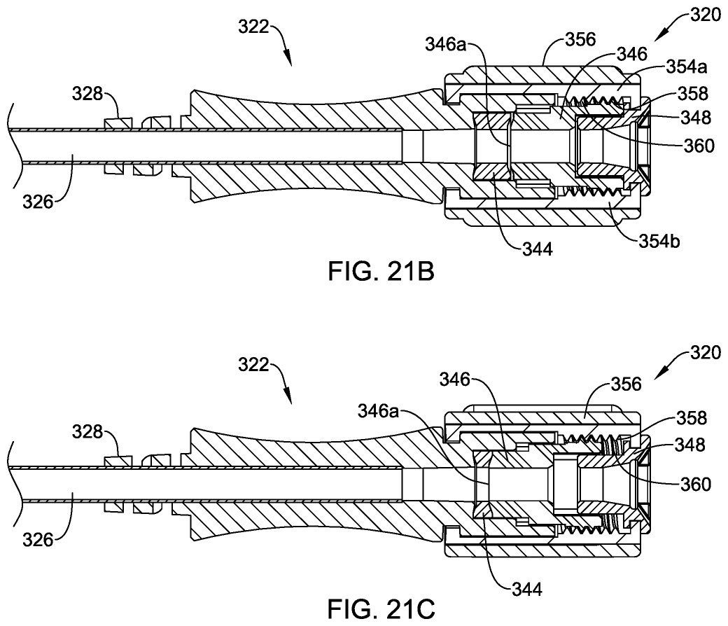

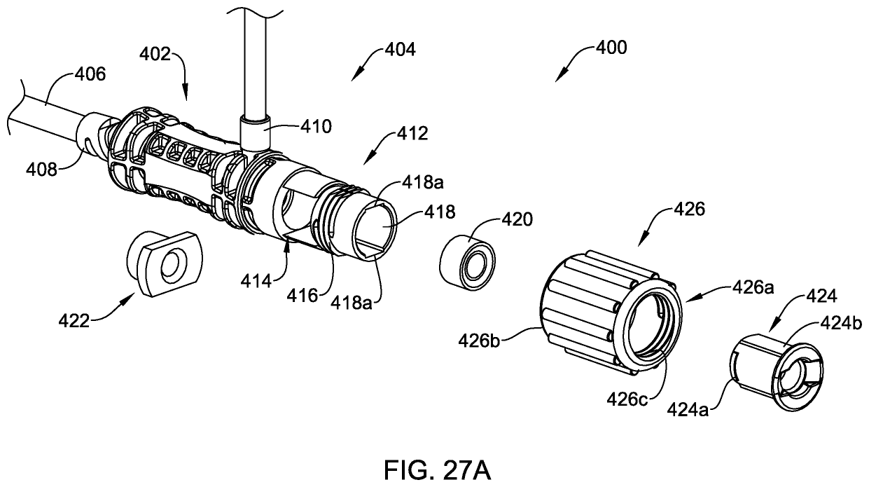

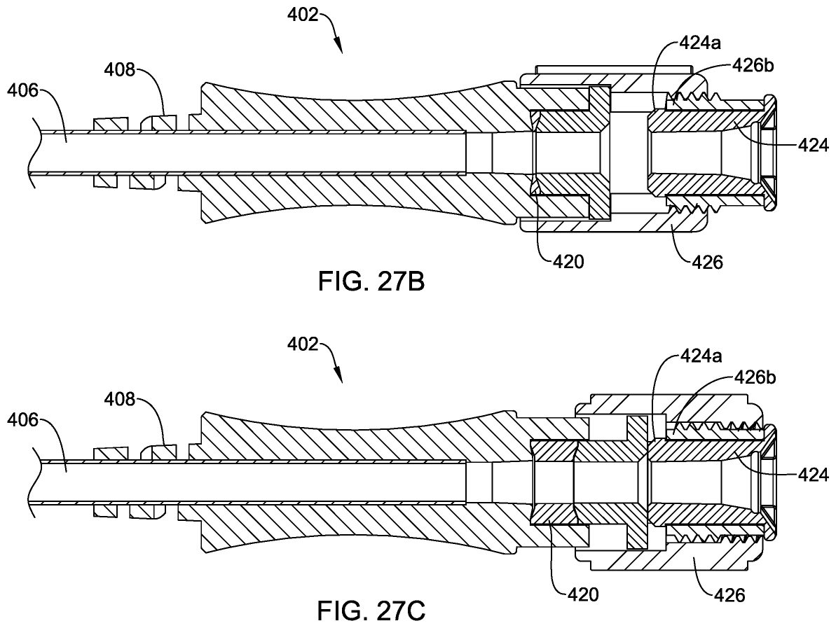

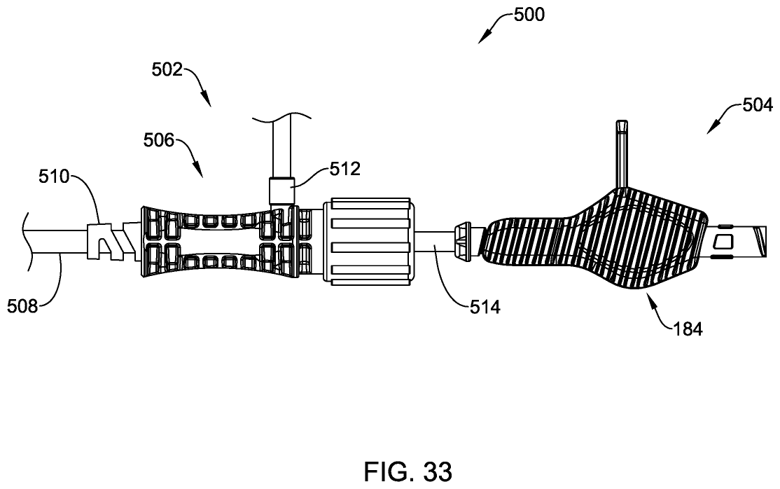

Described herein are a number of mechanisms for providing a releasable rotational lock between a guide catheter such as the guide catheter 32 and an elongate dilator such as the elongate dilator 34. FIGS. 3 through 11B provide views of an Example A for providing a releasable rotational lock between a guide catheter and an elongate dilator. FIGS. 12 through 20 provide views of an Example B for providing a releasable rotational lock between a guide catheter and an elongate dilator. FIGS. 21 through 26B provide views of an Example C for providing a releasable rotational lock between a guide catheter and an elongate dilator. FIGS. 27A through 32C provide views of an Example D providing a releasable rotational lock and a releasable axial lock between a guide catheter and an elongate dilator. FIGS. 33 through 35B provide views of an Example E providing a releasable rotational lock between a guide catheter and an elongate dilator.

### Example A

A goal of Example A is to provide a releasable rotational lock between a guide catheter and an elongate dilator, so user does not have to hold onto both the dilator and the guide catheter for rotational alignment. In some instances, this can include a rotational lock between a valve nut and a guide catheter hub, and between the elongate dilator and the valve nut. FIG. 3 is a perspective view of a proximal hub 138 of a guide catheter 132 and a valve nut 144 that is adapted to be secured relative to the proximal hub 138 of the guide catheter 132.

The guide catheter 132 includes a proximal hub 138, one or more ports or fittings 142 extending from the proximal hub 138 (one is shown), and a strain relief 140 extending proximally from the proximal hub 138. An elongate shaft 136 extends proximally from the proximal hub 138, through the strain relief 140. The proximal hub 138 and the valve nut 144 may each include features that provide a releasable rotational lock between the proximal hub 138 (and hence the guide catheter 132) and the valve nut 144.

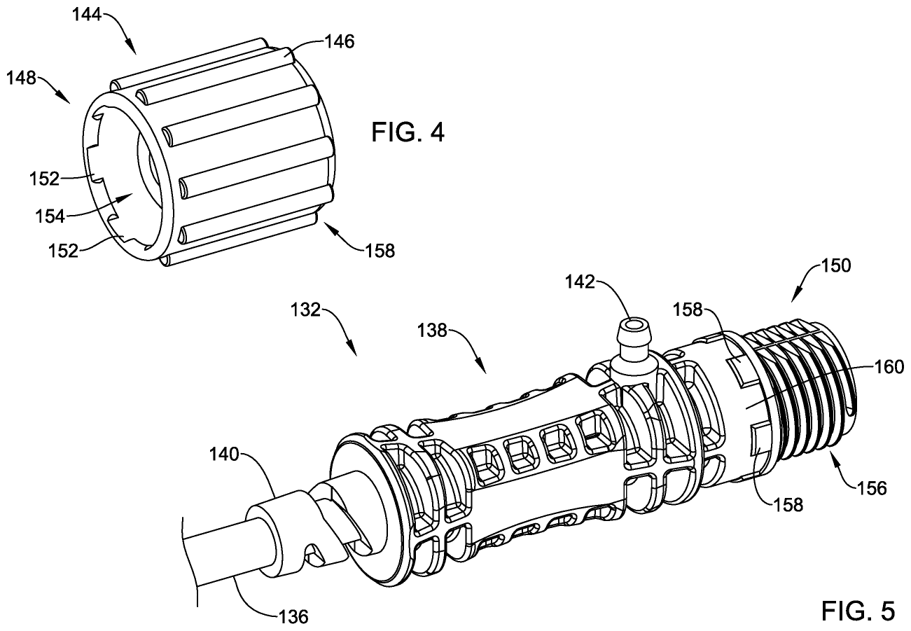

FIG. 4 is a perspective view of the valve nut 144, illustrating features that allow for a releasable rotational lock between the valve nut and the proximal hub. The valve nut 144 includes an exterior surface 146 that is knurled to make it easier for the user to manipulate the valve nut 144. A distal region 148 of the valve nut 144 is adapted to fit over a proximal region 150 of the proximal hub 138. The distal region 148 of the valve nut 144 includes a number of notches 152 that are cut into or otherwise formed within the distal region 148 of the valve nut 144. In some cases, the distal region 148 of the valve nut 144 may include a total of six notches 152, each spaced circumferentially about the distal region 148 of the valve nut 144 such that each of the notches 152 have center points that are circumferentially spaced about 60 degrees from neighboring notches 152. In some cases, there may be more than six notches 152 equally spaced about the distal region 148 of the valve nut 144. In some cases, there may be less than six notches 152 equally spaced about the distal region 148 of the valve nut 144. In some instances, each of the six notches 152 have side walls that are angled at about 60 degrees, although other angles are contemplated. As will be discussed, these notches accommodate corresponding raised segments that are formed on the proximal hub (shown for example in FIG. 5). The distal region 148 of the valve nut 144 includes a recessed region 154 that is adapted to fit over the proximal region 150 of the proximal hub 138.

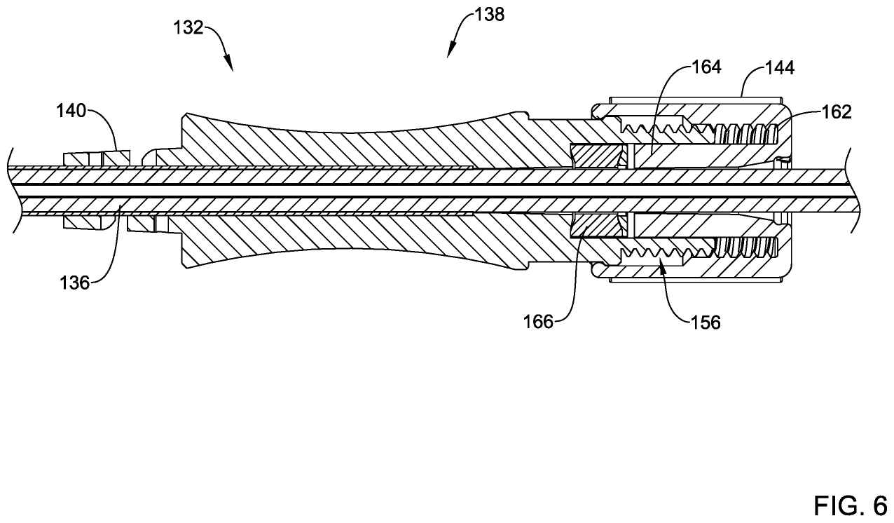

FIG. 5 is a perspective view of the proximal hub 138, illustrating features that allow for a releasable rotational lock between the valve nut 144 and the proximal hub 138. The proximal region 150 of the proximal hub 138 includes an outer threaded surface 156 that is adapted to engage with a corresponding inner threaded surface (surface 162 in FIG. 6) within a proximal region 158 of the valve nut 144. The engagement between the outer threaded surface 156 and the inner threaded surface of the valve nut 144 means that rotating the valve nut 144 relative to the proximal hub 138 may cause axial movement of the valve nut 144 relative to the proximal hub 138.

In some instances, the proximal region 150 of the proximal hub 138 includes a number of tabs 158 that are secured to an outer surface 160 of the proximal region 150. In some cases, the tabs 158 may be integrally molded as part of the proximal hub 138. In some cases, the tabs 158 may be separately formed and subsequently secured to the outer surface 160. The tabs 158 may be adhesively secured, for example. In some cases, there may be a total of six tabs 158, circumferentially spaced about the outer surface 160. In some cases, there may be more than six tabs 158. In some cases, there may be fewer than six tabs 158.

In viewing FIG. 4 and FIG. 5, it will be appreciated that the tabs 158 shown in FIG. 5 are adapted to interact with the notches 152 shown in FIG. 4. In some cases, the tabs 158 have sides that are angled at about 60 degrees. In some cases, the tabs 158 may have sides that are angled appropriately to match or at least substantially match the angled sides of the notches 152. In some cases, the number of tabs 158 will match the number of notches 152. While a total of six tabs 158 are shown on the proximal hub 132 and a total of six notches 158 are shown on the distal region 148 of the valve nut 144, this is merely illustrative. In some cases, there may be less than six tabs 158 and less than six notches 152. In some cases, there may be more than six tabs 158 and more than six notches 152. In some cases, there may be two tabs 158 and four notches 152, or perhaps three tabs 158 and six notches 152, provided that the circumferential spacing between the tabs 158 is a multiple of the circumferential spacing between the notches 152. In any event, the tabs 158 and notches 152 may cooperate to provide a releasable rotational lock between the proximal hub 138 and the valve nut 144.

FIG. 6 is a schematic cross-sectional view. As shown in FIG. 6, the notches 152 formed in the proximal region 148 of the valve nut 144 will engage with the tabs 158 on the proximal region 150 of the proximal hub 132 as the user retracts the valve nut 144. In some cases, the user will feel the rotational lock engage. Because the notches 152 and the tabs 158 both have sides that in some cases are angled at about 60 degrees, the notches 152 and tabs 158 together will provide a rotational lock.

As shown in FIG. 6, an inner threaded surface 162 within the valve nut 144 has not yet reached and engaged the corresponding outer threaded surface 156 of the proximal hub 138. The valve nut 144 includes an inner protruding segment 164 that extends distally from the inner threaded surface 162. Depending on the relative position of the valve nut 144 relative to the proximal hub 138, the inner protruding segment 164 may engage a valve 166 that is disposed within the proximal hub 138. As an example, the rotational lock may resist an applied torque of about 4.67 N-cm (0.41 lb-in). In some cases, applying sufficient torque to the valve nut 144 relative to the proximal hub 138 may be sufficient to overcome the rotational lock, if necessary. FIG. 6 shows the valve nut 144 retracted from the valve 166. In some instances, the valve 166 may be an elastomeric member, for example.

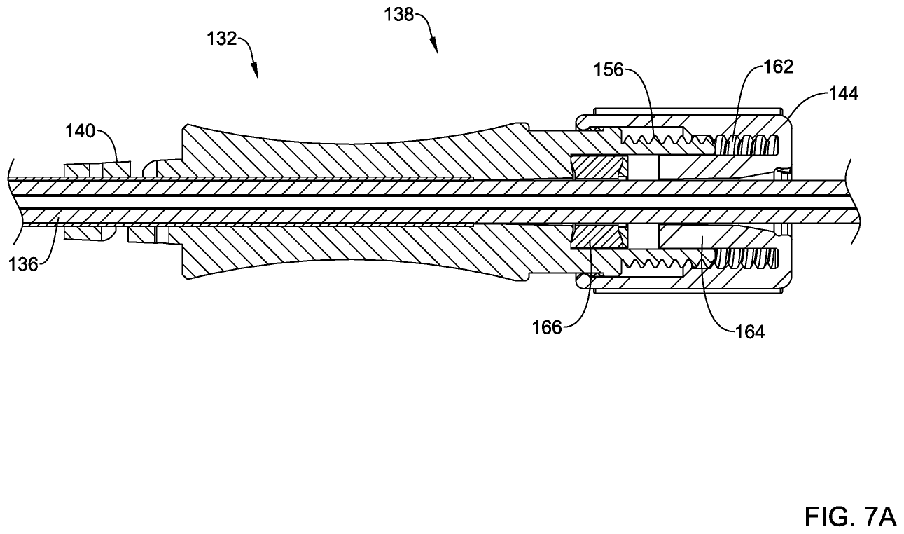

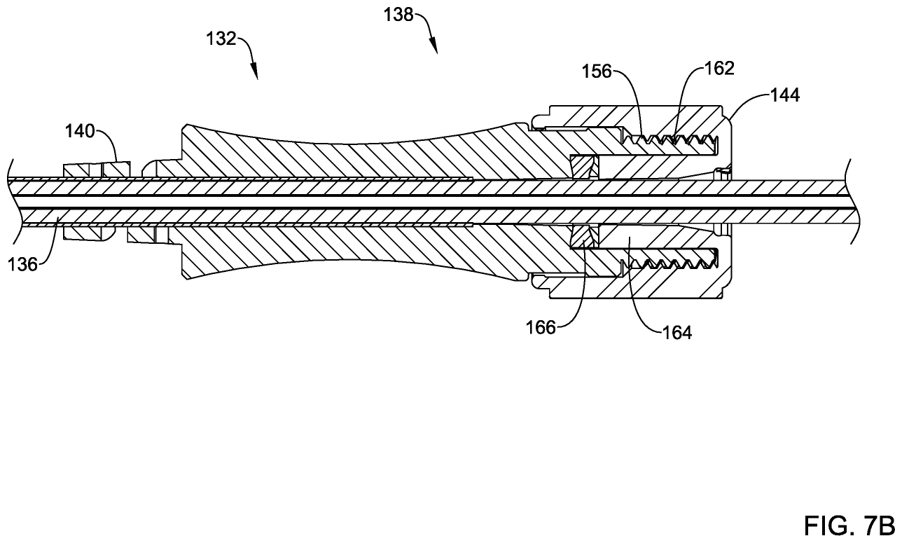

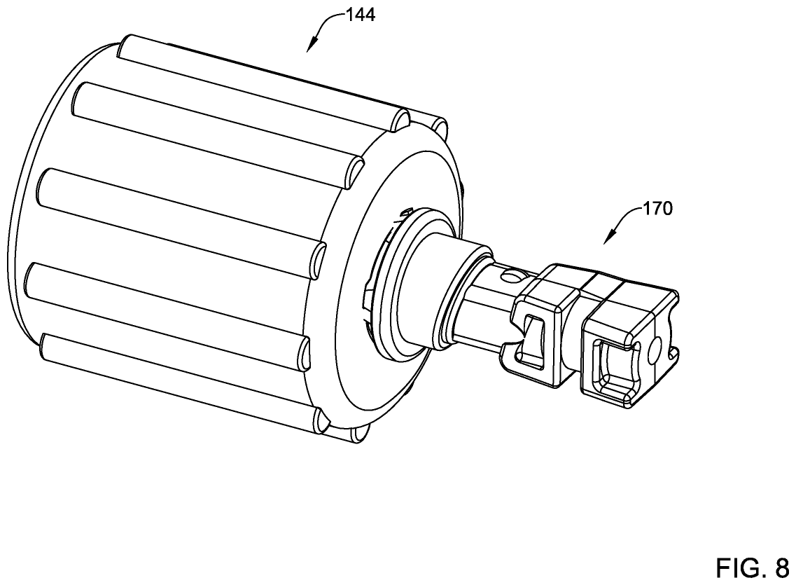

In FIG. 7A, the user has advanced the valve nut 144 sufficiently for the notches 152 to have disengaged from the tabs 158. The user may feel a smooth rotation once the notches 152 have disengaged from the tabs 158. In FIG. 7A, the notches 152 and the tabs 158 have reached the limit of engagement. The valve 166 is just about in contact with the inner protruding segment 164 of the valve nut 144, but the inner protruding segment 164 of the valve nut 144 has not yet started to compress the valve 166. In FIG. 7B, the valve nut 144 has moved farther, and the inner threaded surface 164 of the valve nut 144 has engaged the corresponding inner threaded surface 156 of the proximal hub 138. As can be seen, the tabs 158 on the proximal hub 130 do not interfere with normal valve function. As can be seen, the inner protruding segment 164 of the valve nut 144 has substantially compressed the valve 166.

The previous Figures illustrate a releasable rotational lock between the proximal hub 138 and the valve nut 144. In order to provide a releasable rotational lock between the guide catheter 132 and an elongate dilator, it is useful to also provide a releasable rotational lock between the valve nut 144 and the elongate dilator. FIG. 8 is a perspective view showing a connection between the valve nut 144 and a snap fitting 170 that forms a distal region of an elongate dilator. The snap fitting 170 is adapted to accommodate an elongate shaft extending therethrough, and may be adapted to fit into a dilator hub.

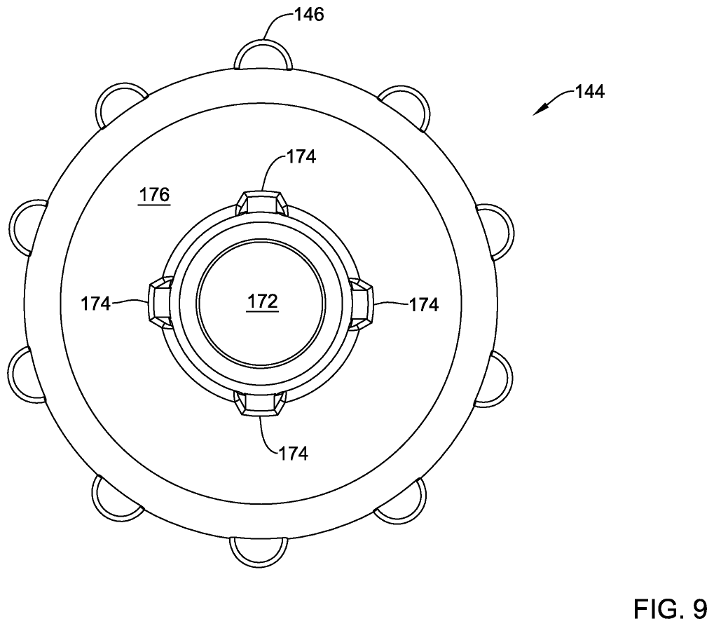

As seen in particular in FIG. 9, which is a proximal end view of the valve nut 144, the valve nut 144 includes an aperture 172 that is adapted to receive a portion of an elongate dilator. In some cases, the aperture 172 is dimensioned to receive a dilator shaft (not shown) extending distally from a dilator hub (such as the dilator hub 46 shown in FIGS. 2A and 2B). In some instances, the valve nut 144 includes several slots 174 that are formed in a proximal end surface 176 of the valve nut 144. In some cases, the slots 174 may be equally spaced about a periphery of the aperture 172. If there are a total of four slots 174, for example, the four slots 174 may be spaced about 90 degrees apart around the periphery of the aperture 172. In some cases, there may be more than four slots 174. In some cases, there may be fewer than four slots 174. The knurled outer surface 146 of the valve nut 144 can also be seen in FIG. 9.

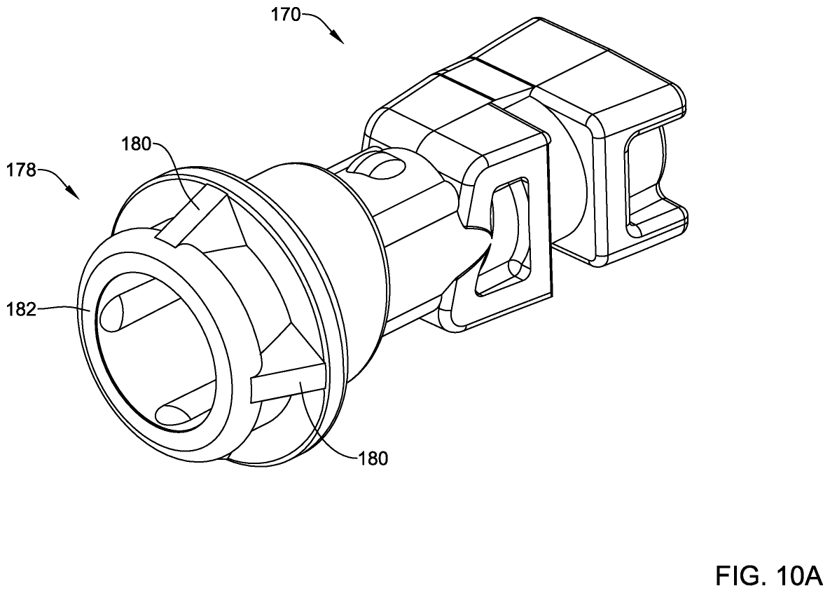

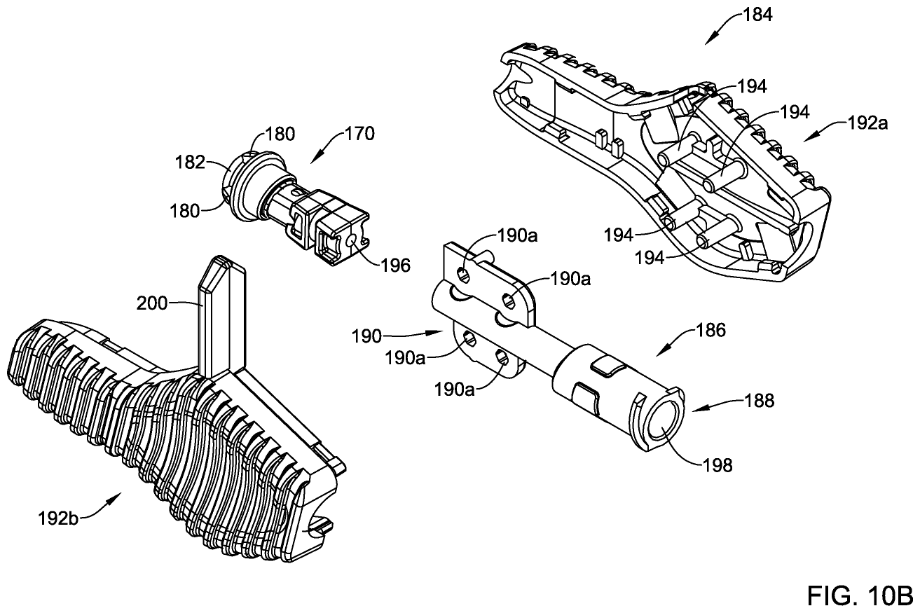

FIG. 10A is a perspective view of the snap fitting 170. A distal region 178 of the snap fitting 170 includes several ribs 180 that are adapted to fit into the slots 174 formed around the aperture 172 of the valve nut 144. In some cases, the distal region 178 of the snap fitting 170 may include several ribs 180 that are equally spaced about the distal region 178 of the snap fitting 170. If there are a total of four ribs 180, for example, the four ribs 180 may be spaced about 90 degrees apart around the periphery of the aperture 172. In some cases, there may be more than four ribs 180. In some cases, there may be fewer than four ribs 180. It will be appreciated that in some instances the slots 174 formed within the proximal end surface 176 of the valve nut 144 will be equal in number and distribution with the corresponding ribs 180 formed within the distal region 178 of the snap fitting 170. In some cases, there may be one rib 180 and four slots 174, or perhaps two ribs 180 and four slots 174, provided that the circumferential spacing between the tabs 158 is a multiple of the circumferential spacing between the notches 152. As shown, a total of four slots 174 have been added but do not interfere with the snap geometry of the valve nut 144. FIG. 10A shows that the snap fitting 170 includes a full round snap profile 182.

FIG. 10B is an exploded view of an illustrative dilator hub 184, showing how the snap fitting 170 forms part of the dilator hub 184. The dilator hub 184 may be used as part of an elongate dilator that is adapted to have a releasable rotational lock with the valve nut 144. The dilator hub 184 includes a Luer fitting 186 having a distal region 188 providing a Luer fitting and a proximal region 190 that includes several apertures 190a. The dilator hub 184 includes a first clamshell 192a and a second clamshell 192b that together form a graspable portion of the dilator hub 184. The first clamshell 192a includes several protrusions 194 that are adapted to extend through the apertures 190a formed in the Luer fitting 186 and extend into corresponding apertures (not visible in this view) formed within the second clamshell 192b. In some instances, the protrusions 194 form a snap fitting with the corresponding apertures formed within the second clamshell 192b. As the first clamshell 192a is fitted to the second clamshell 192b, the snap fitting 170 and the Luer fitting 186 are entrapped between the first clamshell 192a and the second clamshell 192b. An elongate shaft forming part of the elongate dilator is not shown, but is accommodated by a lumen 196 extending through the snap fit 170. The lumen 196 may be considered as being in fluid communication with a corresponding lumen 198 extending through the Luer fitting 186. The second clamshell 192b includes an extension 200 that is adapted to provide an indication of a direction of curvature for a distal region of the elongate shaft.

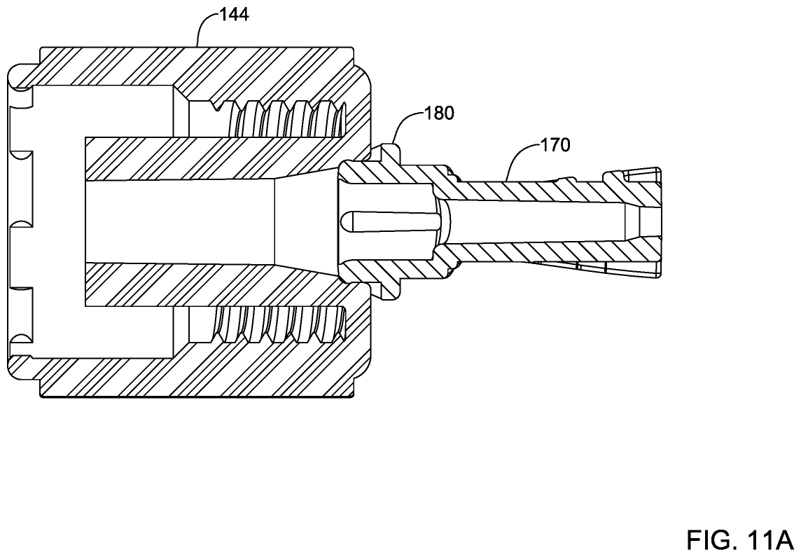

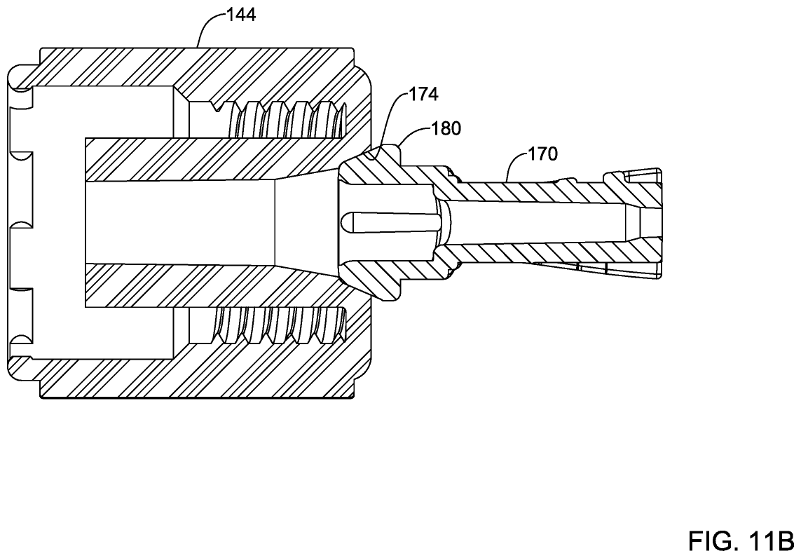

FIG. 11A is a schematic cross-sectional view showing engagement of the round snap profile 182. FIG. 11B, which represents a small rotation of snap fitting 170 relative to the valve nut 144, shows several ribs 180 fitting into corresponding slots 174, thereby providing a releasable rotational lock between the valve nut 144 and the snap fitting 170 (and hence between the valve nut 144 and the dilator hub 184). With four tabs and four notches, the valve nut 144 can lock into any of four positions, each about 90 degrees apart. This works with the four ribs 180 formed on the snap fitting 170, and may be used to always orient to a particular position which aligns a curve indicator with the side port tube. Because there are other possible positions, it is up to the user to align appropriately when snapping in the elongate dilator. In some cases, as noted, there may be less than four positions, but a minimum of two or three positions seems appropriate to accommodate thread length.

### Example B

A goal of Example B is to provide rotational lock between a guide catheter and an elongate dilator, so that a user does not have to separately hold both components. FIG. 12 is a perspective view showing a valve nut 244 attached to a proximal hub 238 of a guide catheter 232. A valve compressor 246, which as will be discussed fits within the valve nut 244, includes features that provide a smooth funnel for accommodating and accepting an elongate dilator and provides a releasable rotational lock with an elongate dilator.

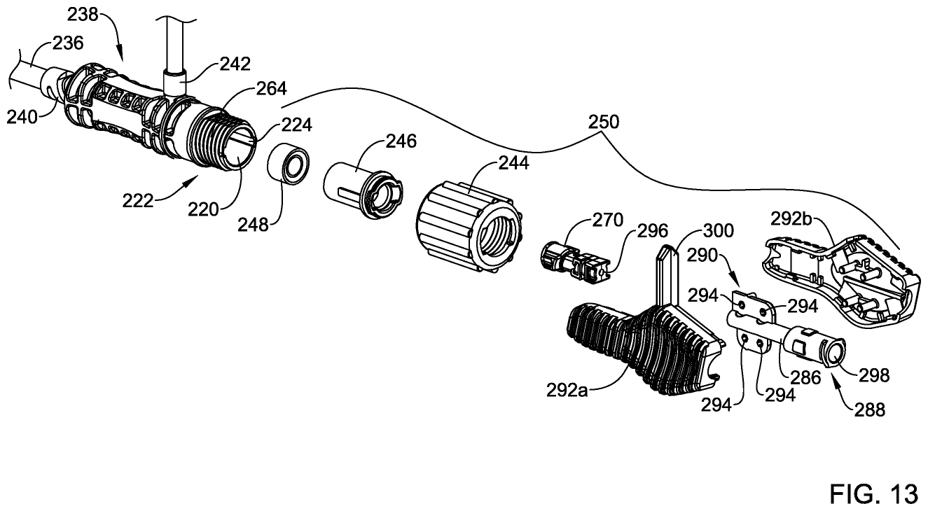

As seen in FIG. 12, the guide catheter 232 includes a proximal hub 238, one or more ports or fittings 242 extending from the proximal hub 238 (one is shown), and a strain relief 240 extending proximally from the proximal hub 238. An elongate shaft 236 extends proximally from the proximal hub 238, through the strain relief 240. FIG. 13 is an exploded view showing the proximal hub 238 of the guide catheter 232 and several components that together form the valve nut 244 and parts of a dilator hub 250. The dilator hub 250 may be combined with an elongate dilator shaft (not shown) in order to form an elongate dilator that may be used in combination with the guide catheter 232. The additional components include a valve 248, the valve compressor 246, the valve nut 244, a snap tip 270 (which may be considered as being similar to the snap fit 170), a universal Luer 286, a first clamshell 292a and a second clamshell 292b. The valve 248, which may be an elastomeric element, fits into an aperture 220 that is formed within a proximal region 222 of the proximal hub 238.

The Luer fitting 286 includes a distal region 288 providing a Luer fitting and a proximal region 290 that includes several apertures 290a. The dilator hub 250 includes a first clamshell 292a and a second clamshell 292b that together form a graspable portion of the dilator hub 250. The first clamshell 292a includes several protrusions 294 that are adapted to extend through the apertures 290a formed in the Luer fitting 186 and extend into corresponding apertures (not visible in this view) formed within the second clamshell 292b. In some instances, the protrusions 294 form a snap fitting with the corresponding apertures formed within the second clamshell 292b. As the first clamshell 292a is fitted to the second clamshell 292b, the snap tip 270 and the Luer fitting 286 are entrapped between the first clamshell 292a and the second clamshell 292b. An elongate shaft forming part of the elongate dilator is not shown, but is accommodated by a lumen 296 extending through the snap tip 270. The lumen 296 may be considered as being in fluid communication with a corresponding lumen 298 extending through the Luer fitting 286. The second clamshell 292b includes an extension 300 that is adapted to provide an indication of a direction of curvature for a distal region of the elongate shaft.

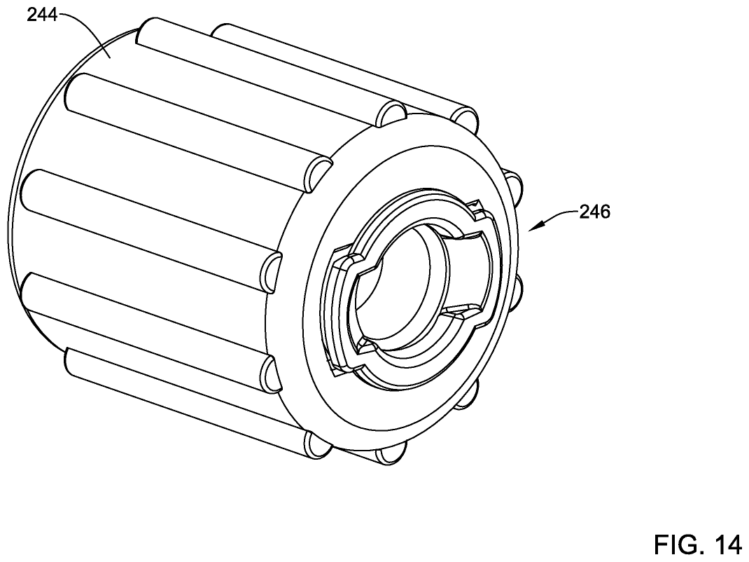

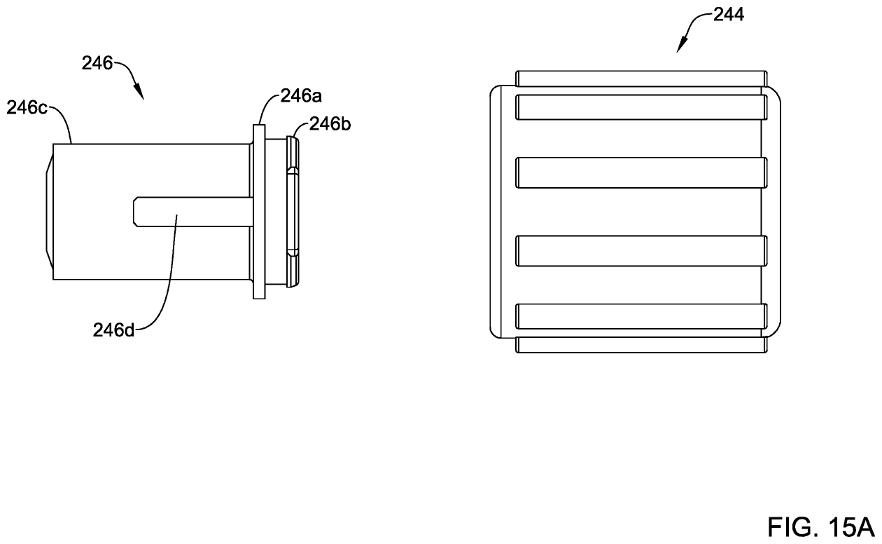

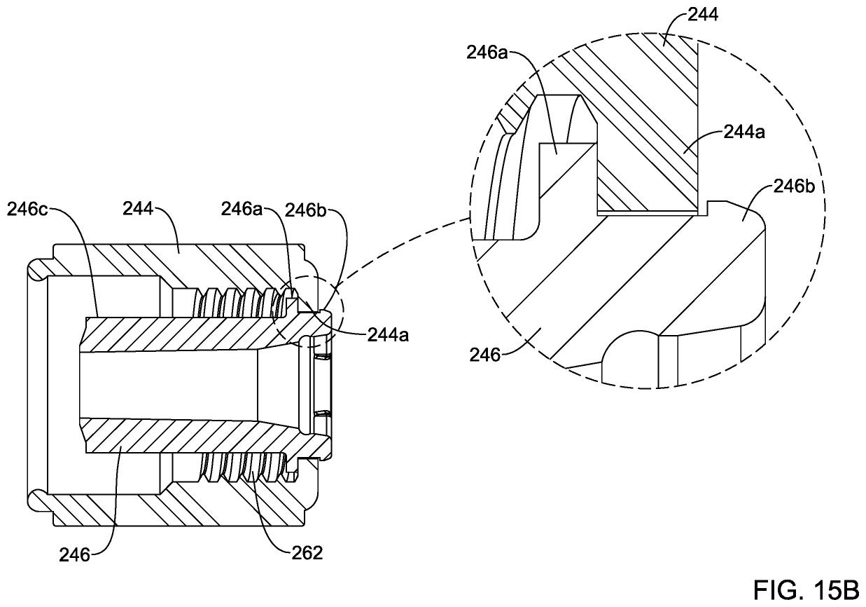

FIG. 14 is a perspective view showing the valve compressor 246 disposed within the valve nut 244. FIG. 15A is an exploded side view of the valve compressor 246 and the valve nut 244 and FIG. 15B is a cross-sectional view thereof. The valve compressor 246 includes a raised ridge 246a and an outer profile 246b that together provides a snap fit between the valve compressor 246 and an annular shoulder 244a of the valve nut 244. This allows the valve compressor 246 to be snapped into the valve nut 244. The annular shoulder 244a is adapted to resist forces created by torque applied to the valve nut 244. It will be appreciated that a proximal portion 246c is adapted to engage the valve 248 and as such may be considered as serving a similar function to the inner protruding segment 164 described with respect to the valve nut 144.

The valve compressor 246 includes several ribs 246d that engage with corresponding slots 224 that are formed within the proximal region 222 of the proximal hub 238. In some cases, the valve compressor 246 may include two ribs 246d that are spaced about 180 degrees apart, and the proximal hub 238 may have two corresponding slots 224. In some instances, the valve compressor 246 may include three or more ribs 246 that are spaced about the valve compressor 246, with a corresponding number of slots 224 within the proximal hub 238.

As a result, the valve compressor 246 does not rotate relative to the proximal hub 238. Rather, the valve nut 244 rotates relative to the proximal hub 238. In some instances, the valve nut 244 and the valve compressor 246 may together be considered as forming a two-piece valve nut, with the valve nut 244 being considered an outer component and the valve compressor 246 being considered an inner component. The valve nut 244 includes an inner threaded surface 262 that is adapted to engage a corresponding outer threaded surface 264 formed within the proximal region 222 of the proximal hub 238.

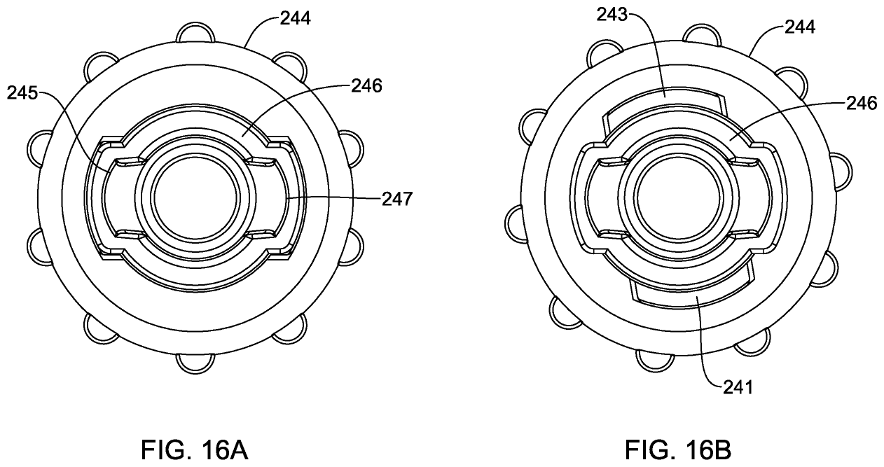

As seen in FIG. 16A, the valve compressor 246 includes ears 245 and 247 that accommodate rotational lock. Because of the ears 245 and 247 forming part of the valve compressor 246, and corresponding notches 241 and 243 that are formed within the valve nut 244, the snap spans two regions that are each about 100 degrees. As shown in FIG. 16B, the ears 245 and 247 need to be aligned with the notches 241 and 243 in the outer nut 244 during assembly, after assembly parts freely rotate.

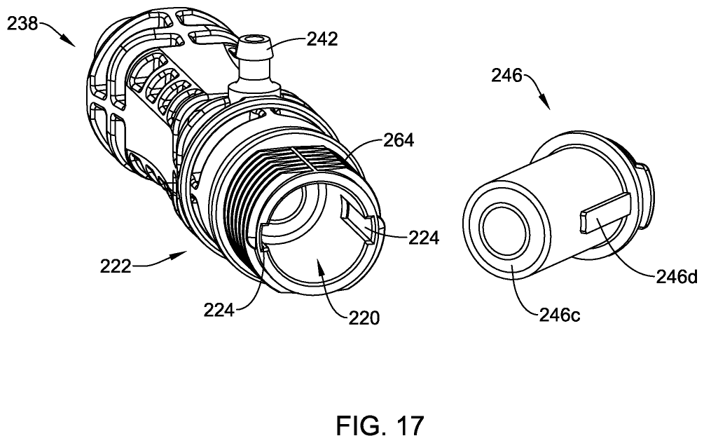

FIG. 17 is a perspective view of the valve compressor 246 relative to the proximal hub 238, providing another view of the ribs 246d that engage with the corresponding slots 224 that are formed within the proximal region 222 of the proximal hub 238. In some instances, the ribs 246d and the slots 224 cooperate to provide a particular orientation of the valve compressor 246 relative to the proximal hub 238 and the fitting 242. It will be appreciated that the relative position of the ribs 246d and the slots 224 are spaced about 90 degrees from the position of the fitting 242. This same spacing is carried through the valve compressor 246 via the relative position of the ears 245 and 247 (of the valve compressor 246) and the notches 241 and 243 (of the valve nut 244). Consequently, the dilator hub 250 will be positioned such that the extension 300 of the clamshell 292a will be aligned with the fitting 242. This means that a curved distal region of an elongate dilator including the dilator hub 250 will be aligned with a corresponding curved distal region of the elongate shaft 236 forming part of the guide catheter 232.

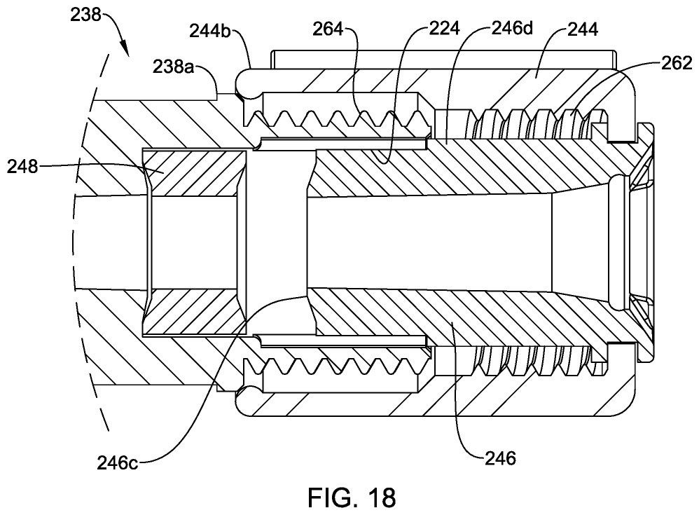

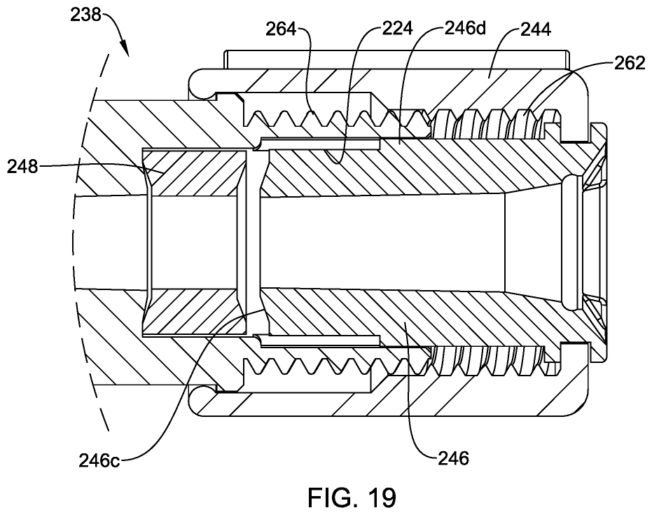

FIGS. 18 and 19 are cross-sectional views showing how the valve nut 244 (carrying the valve compressor therein) snaps onto the proximal hub 238. In some instances, the proximal hub 238 includes an annular ridge 238a that is adapted to engage a corresponding annular snap 244b formed on the valve nut 244. In FIG. 18, the annular snap 244b is positioned adjacent the annular ridge 238a but has not yet been pushed over the annular ridge 238a. In FIG. 19, the annular snap 244b has been pushed over the annular ridge 238a.

Also visible in FIGS. 18 and 19 are the ribs 246d and the slots 224. The ribs 246d need to be aligned with the slots 224 before the valve nut 244 can be pushed from the position relative to the proximal hub 238 shown in FIG. 18 to the position relative to the proximal hub 238 shown in FIG. 19. In FIG. 18, the inner threaded surface 262 of the valve nut 244 has not yet engaged the outer threaded surface 264 formed on the proximal hub 238. In FIG. 19, the inner threaded surface 262 of the valve nut 244 has just engaged the outer threaded surface 264 formed on the proximal hub 238. As can be seen, the slots 224 stop before the location of the valve 248, such that valve function is not affected by the slots 224.

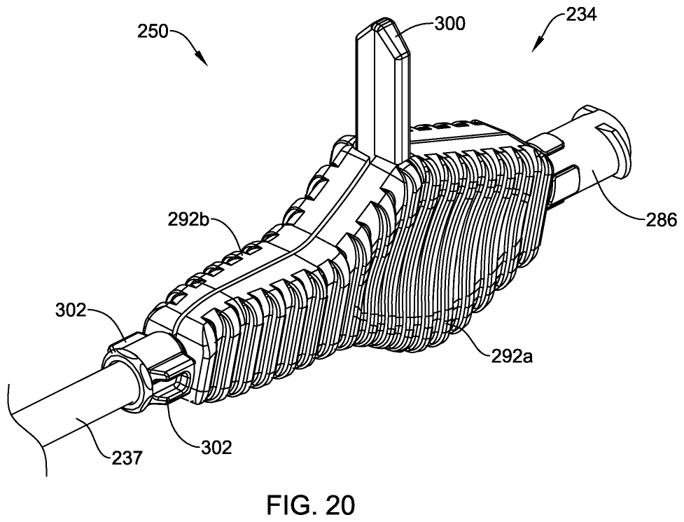

FIG. 20 is a perspective view of an illustrative elongate dilator 234 that includes the dilator hub 250. An elongate dilator shaft 237 extends proximally from the clamshell 292a and the clamshell 292b, here snapped together to form the dilator hub 250. The dilator hub 250 includes several ribs 302 that are adapted to fit into the ears 245 and 247 formed within the valve compressor 246. In some cases, the ribs 302 are formed as part of the snap tip 270, for example. As shown, the valve compressor 246 includes two ears 245 and 247 and the snap tip 270 includes two corresponding ribs 302. In some cases, the valve compressor 246 may include three or more ears, and the snap tip 270 may correspondingly include three or more ribs 302. In some cases, the valve compressor 246 may include a single ear and the snap tip 270 may include a single rib 302.

### Example C

A goal of Example C is to provide a rotational and axial lock between a guide catheter and an elongate dilator, so that a user does not have to separately hold both components. FIG. 21A is an exploded view, showing an assembly 320 that includes a proximal hub 322. A fitting 324 extends from the proximal hub 322 and may be used to supply a fluid to interior of the proximal hub 322. An elongate shaft 326 extends proximally from the proximal hub 322, and includes a strain relief 328 that extends over the elongate shaft 326 a short distance. In some instances, the position of the fitting 324 may provide an indication of a direction of curvature when the elongate shaft 326 includes a curved distal region (not shown). A proximal region 340 of the proximal hub 322 does not include an outer threaded surface. An aperture 342 is formed within the proximal region 340 of the proximal hub 322.

The assembly 320 includes a valve 344 that may be an elastomeric member, for example, and that fits into the aperture 342. A compressor 346 is adapted to translate relative to the valve 344 in order to reversibly compress the valve 344. A snap 348 is adapted to fit within an aperture 350 formed within the compressor 346. The snap 348 includes an aperture 352 that is adapted to accommodate an elongate dilator. The assembly 320 includes a first nut half 354a and a second nut half 354b. A nut sleeve 356 fits over the first nut half 354a and the second nut half 354b in order to hold the first nut half 354a and the second nut half 354b together. The first nut half 354a and the second nut half 354b together define an inner threaded surface 358 that is adapted to engage a corresponding outer threaded surface 360 that is formed on the compressor 346. This allows the compressor 346 to translate relative to the first nut half 354a and the second nut half 354b.

FIGS. 21B and 21C are cross-sectional views showing how the compressor 346 translates in order to compress the valve 344 while the snap 348 remains stationary. In FIG. 21B, the compressor 346 has contacted the valve 344 but has not translated far enough to compress the valve 344. In FIG. 21C, the compressor 346 has translated farther, and has substantially compressed the valve 344. A distal end 346a of the compressor 346 is adapted to have a shape that is complementary to that of the valve 344. In some instances, this may mean that no washer is needed. It can be seen that the inner threaded surface 358 defined by the first nut half 354a and the second nut half 354b engages the outer threaded surface 360 formed on the compressor 346 in order to allow rotation of a nut assembly including the first nut half 354a, the second nut half 354b and the nut sleeve 356 to cause the compressor 346 to translate.

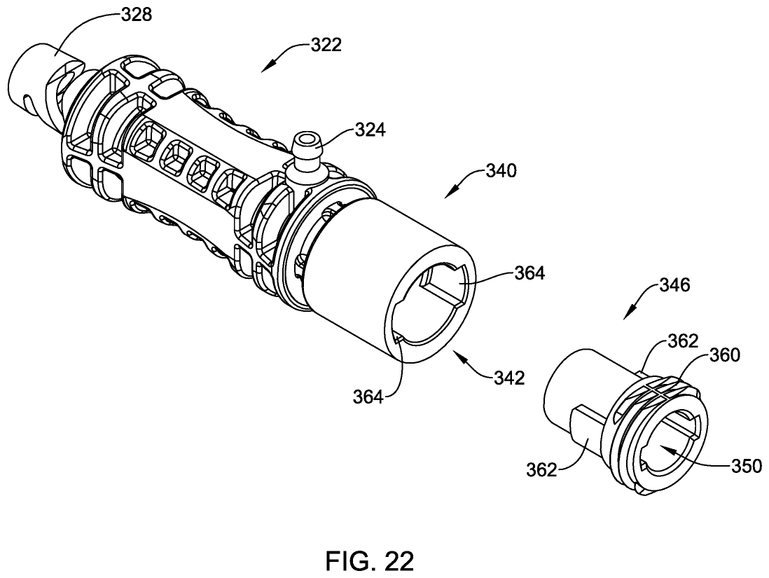

FIG. 22 is a perspective view showing the compressor 346 aligned for insertion into the aperture 342 formed within the proximal region 340 of the proximal hub 322. The compressor 346 includes several ribs 362 that are adapted to extend into several corresponding slots 364 that are formed within the aperture 342. It will be appreciated that engagement of the ribs 362 with the slots 364 will allow the compressor 346 to translate relative to the proximal hub 322 but will not allow the compressor 346 to rotate relative to the proximal hub 322. In some cases, the ribs 362 only extend a fraction of a length of the compressor 346. While a total of two ribs 362 and two slots 364 are shown, in some cases there may be three or more ribs 362 and a corresponding three or more slots 364. In some cases, the compressor 346 may include a single rib 362 and the aperture 342 may include a single slot 364.

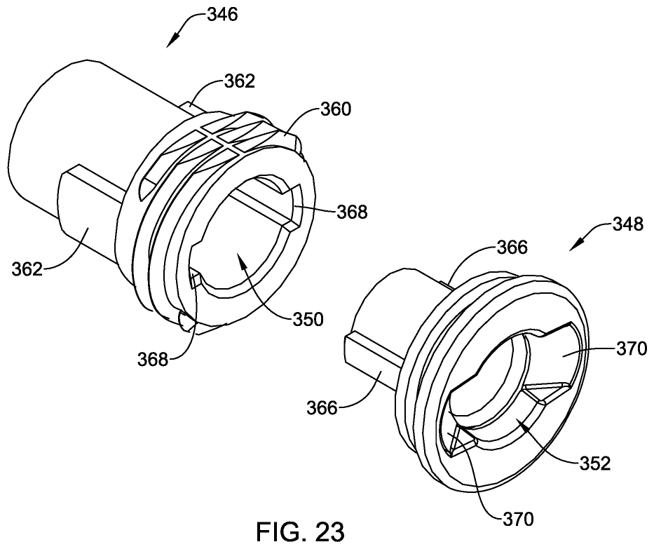

FIG. 23 is a perspective view showing the snap 348 aligned for insertion into the aperture 350 formed within the compressor 346. The snap 348 includes several ribs 366 that are adapted to extend into several corresponding slots 368 that are formed within the aperture 350. It will be appreciated that engagement of the ribs 366 with the slots 368 will allow the compressor 346 to translate relative to the snap 348 but will not allow the compressor 346 to rotate relative to the snap 348. In some cases, the ribs 362 only extend a fraction of a length of the compressor 346. While a total of two ribs 362 and two slots 364 are shown, in some cases there may be three or more ribs 362 and a corresponding three or more slots 364. In some cases, there may be a single rib 362 and a single slot 364. Because the compressor 346 is not allowed to rotate relative to the proximal hub 322, and because the snap 348 is not allowed to rotate relative to the compressor 346, rotational alignment between the proximal hub 322 and the snap 348 is maintained.

It will be appreciated that the aperture 352 formed within the snap 352 includes ears 370 that are adapted to accommodate the ribs on a dilator hub of an elongate dilator in order to maintain a rotational orientation between the proximal hub 322 and the elongate dilator. In some instances, this rotational orientation extends to aligning a curved distal region of an elongate dilator with a corresponding curved distal region of the elongate shaft 326 of a guide catheter.

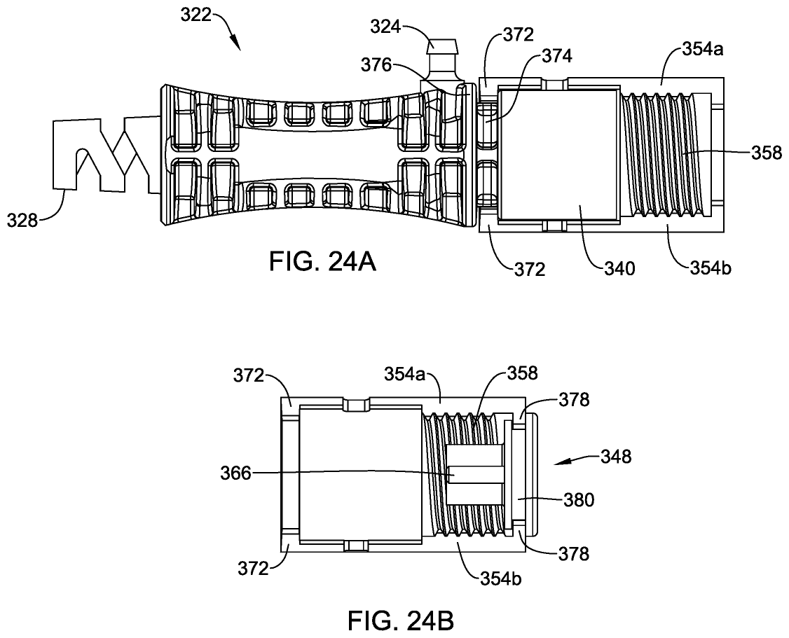

FIG. 24A is a side view showing how the first nut half 354a and the second nut half 354b interact with the proximal portion 340 of the proximal hub 322. The first nut half 354a and the second nut half 354b each define part of an annular flange 372 that is adapted to engage a narrowed portion 374 of the proximal portion 340 of the proximal hub 322. The narrowed portion 374 is defined just proximal of an increased diameter portion 376. The first nut half 354a and the second nut half 354b are able to rotate freely around the proximal hub 322. The engagement between the annular flange 372 and the narrowed portion 374 provides an axial lock between the first nut half 354a, the second nut half 354b and the proximal hub 322.

FIG. 24B is a side view showing how the snap 348 interacts with the first nut half 354a and the second nut half 354b. The first nut half 354a and the second nut half 354b together define an annular flange 378 that is adapted to engage an annular recessed portion 380 that is formed within the snap 348. The engagement between the annular flange 378 and the annular recessed portion 380 constrains the snap 348 axially but allows the first nut half 354a and the second nut half 354b to rotate freely relative to the snap 348.

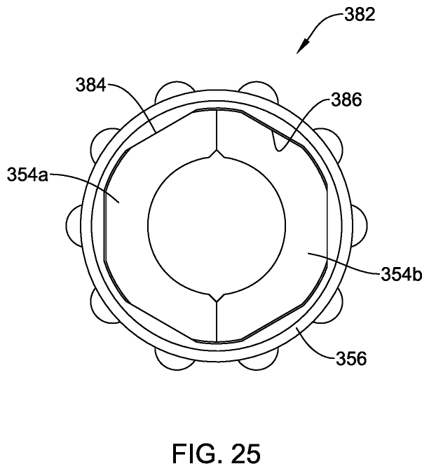

FIG. 25 is an end view of a valve nut assembly 382. The valve nut assembly 382 includes the first nut half 354a and the second nut half 354b, constrained within the nut sleeve 356. In some cases, the nut sleeve 356 may be pressed onto the first nut half 354a and the second nut half 354b. In some instances, the nut sleeve 356 may snapped over the first nut half 354a and the second nut half 354b. In some cases, the nut sleeve 356 may be glued onto the first nut half 354a and the second nut half 354b. The nut sleeve 356 prevents the first nut half 354a and the second nut half 354b from separating. A number of flattened surfaces 384 that are formed on outer surfaces of the first nut half 354a and the second nut half 354b interact with corresponding flattened surfaces 386 that are formed on an inner surface of the nut sleeve 356 in order to provide for torque transmission between the nut sleeve 356 and the first nut half 354a and the second nut half 354b. This means that rotating the nut sleeve 356 will cause a corresponding rotation in the first nut half 354a and the second nut half 354b.

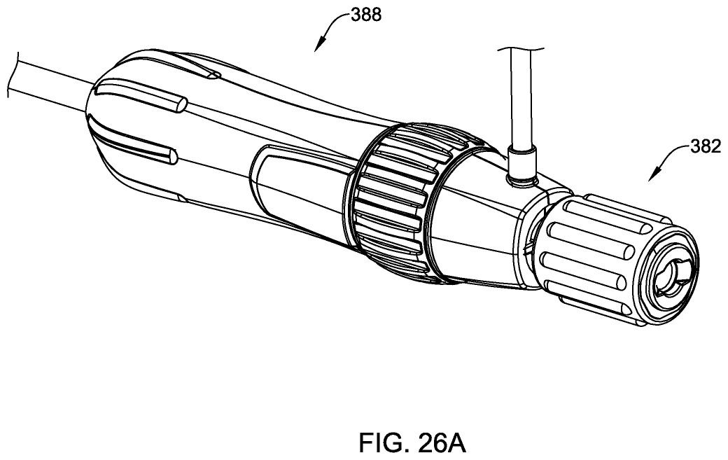

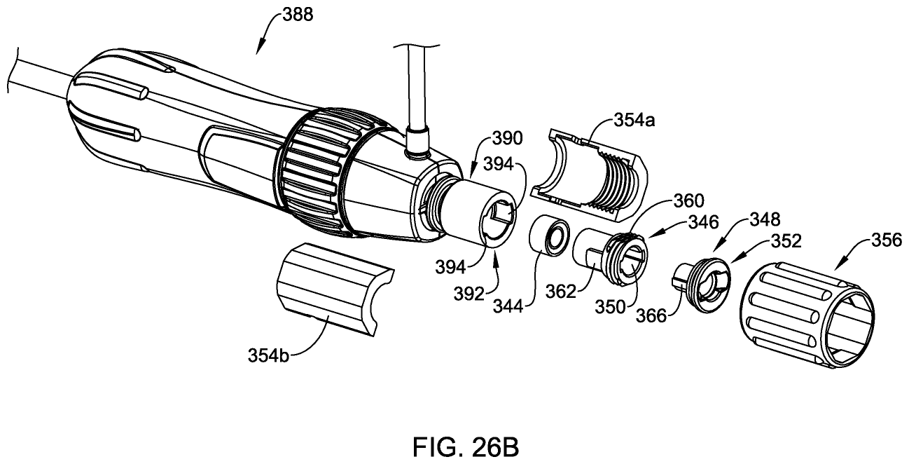

FIG. 26A is a perspective view showing a WATCHMAN TruSteer™ Access Sheath hub 388 in combination with the valve nut assembly 382. TruSteer is a steerable access device that is described, for example, in U.S. Provisional Application Ser. No. 63/316,208, which application is incorporated by reference herein. As shown in FIG. 26B, which is an exploded view thereof, the hub 388 includes a proximal portion 390 that is similar to that of the proximal hub 322. An aperture 392 formed in the proximal portion 390 of the hub 388 includes slots 394 that are adapted to accommodate the ribs 362 formed on the compressor 346.

### Discussion of Example D

A goal of Example D is to provide a rotational and axial lock between the guide catheter and the elongate dilator, so that a user does not have to separately hold both components. FIG. 27A is an exploded view of an assembly 400, showing a proximal hub 402 of a guide catheter 404. The guide catheter 404 includes an elongate shaft 406 that extends proximally from the proximal hub 402. A strain relief 408 extends proximally over the elongate shaft 406 a short distance from the proximal hub 402. The proximal hub 402 includes a proximal portion 412. The proximal portion 412 includes a side window 414, an outer threaded surface 416 and an end aperture 418.

The assembly 400 also includes a valve 420, a compressor 422, a snap 424 and a valve nut 426. In some cases, Example D does not need a washer. The valve 420 may be placed within the end aperture 418, and the compressor 422 may be added behind the valve 420 by inserting the compressor 422 through the side aperture 414. In some instances, the proximal region 412 of the proximal hub 402 includes several slots 418a that interact with corresponding ribs 424b that are formed on the snap 424 in order to provide a rotational alignment between the proximal hub 402 and the snap 424.

FIGS. 27B and 27C are schematic cross-sectional views showing the compressor 422 disposed adjacent the valve 420. The snap 424 fits into an aperture 418 formed within the proximal region 412 of the proximal hub 402. The snap 424 includes several raised segments 424a (one is visible in this view) that interact with a distal edge of the proximal hub 402 in order to secure the snap 424 in place. As can be seen, the snap 424 remains stationary while the compressor 422 translates distally in order to compress the valve 420 (and translates proximally in order to allow the valve 420 to relax. The outer threaded surface 416 of the proximal hub 402 engages a corresponding inner threaded surface 426c of the valve nut 426. A distal end 424c of the snap is adapted to contact the compressor 422.

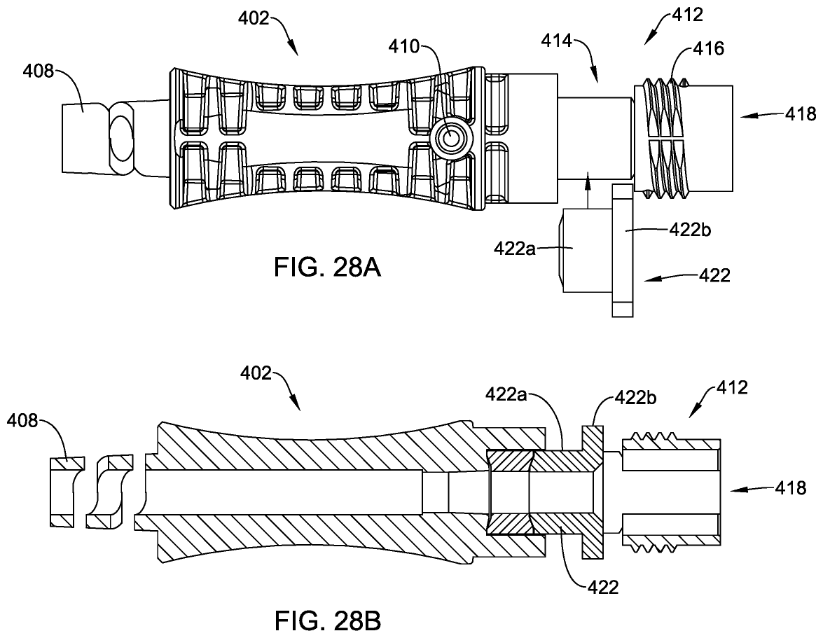

FIG. 28A is a schematic cross-sectional view that shows how the compressor 422 is inserted into the proximal hub 402 through the side window 414. The side window 414 may have a height that matches an ID (inner diameter) of the proximal hub 402 in order to accommodate a full diameter compressor. In some cases, the compressor 422 includes a recessed diameter segment 422a and a full diameter segment 422b. As seen in FIG. 28B, the compressor 422 advances distally in order to actuate the valve 420.

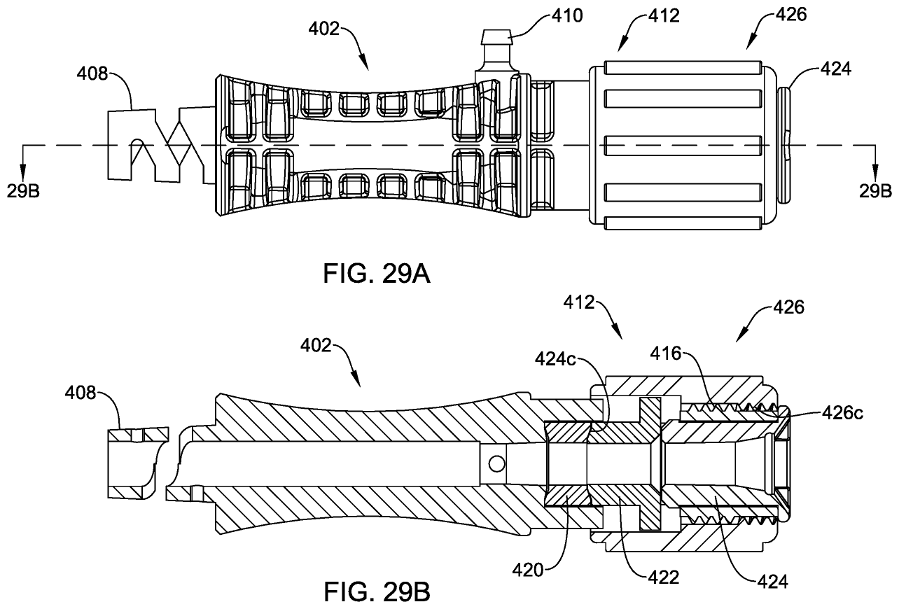

FIG. 29A is a schematic cross-sectional view that shows the valve nut 426 positioned relative to the proximal region 412 of the proximal hub 402. As seen, there is no undercut snap formed in the valve nut 426 in order to retain the valve nut 426 on the proximal hub 402. Rather, a proximal flange of the snap 424 retains the valve nut 426. The valve nut 426 may be constrained radially at both ends to prevent tipping, which if not prevented could cause cross-threading. As can be seen in FIG. 29B, which is a cross-sectional view taken along the line 29B-29B of FIG. 29A, when the valve nut is in a proximal position (as shown in FIG. 29B), the outer threaded surface 416 of the proximal hub 402 does not engage with the inner threaded surface 426c of the valve nut 426. As a result, this allows the valve nut 426 to free spin when the valve 420 is open.

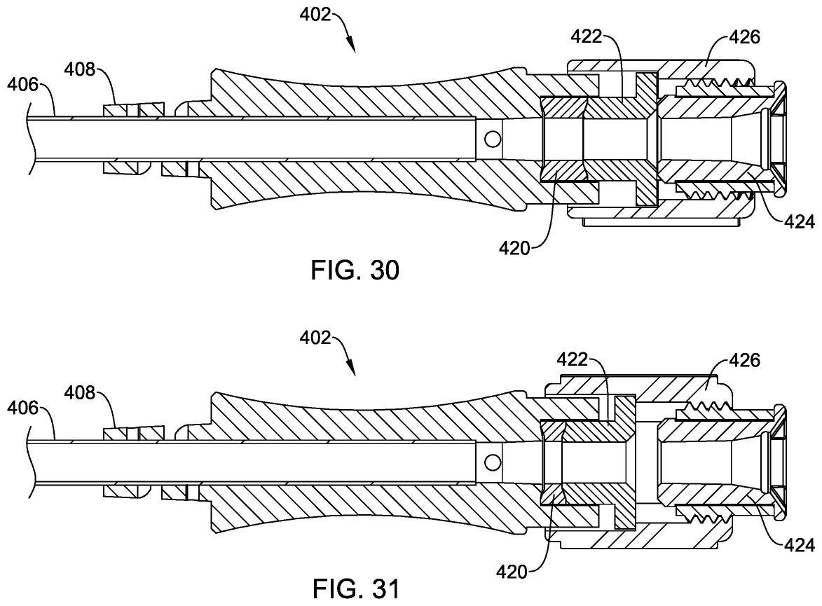

FIGS. 30 and 31 are additional cross-sectional views. The valve nut 426 contacts the compressor 422. When advancing and rotating the valve nut 426 to close the valve 420, the outer threaded surface 416 of the proximal hub 402 engage with the inner threaded surface 426c of the valve nut 426 prior to the valve nut 426 engaging the compressor 422. As a result, the user does not have to apply force in order to compress the valve 420. The valve nut 426 advances the compressor 422 when rotated. The compressor 422 will be returned to the proximal position by the valve 420, not by the valve nut 426.

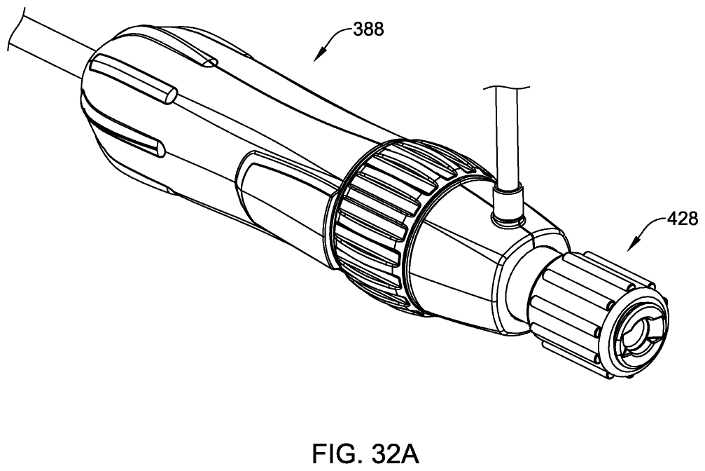

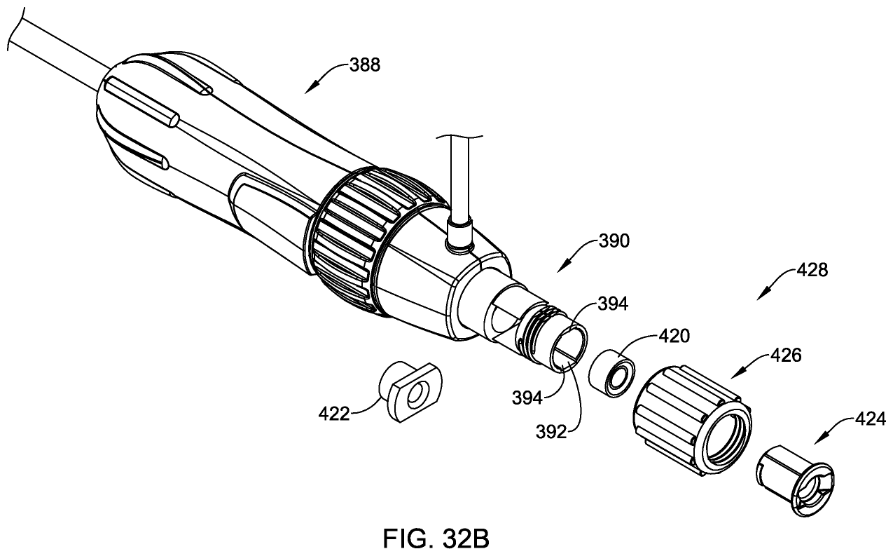

FIG. 32a is a perspective view showing a WATCHMAN TruSteer™ Access Sheath hub 388 in combination with an assembly 428 that includes, in combination, the valve 420, the compressor 422, the snap 424 and the valve nut 426. As shown in FIG. 32B, which is an exploded view thereof, the hub 388 includes a proximal portion 390 that is similar to that of the proximal hub 322. An aperture 392 formed in the proximal portion 390 of the hub 388 includes slots 394 that are adapted to accommodate the ribs 362 formed on the compressor 346.

### Discussion of Example E

A goal of Example E is to provide rotational lock between a guide catheter and an elongate dilator. FIG. 33 is a side view of an illustrative assembly 500 that includes a guide catheter 502 and an elongate dilator 504. The guide catheter 502 includes a proximal hub 506. An elongate shaft 508 extends proximally from the proximal hub 506. A strain relief 510 extends proximally from the proximal hub 506. A fitting 512 enables fluid to be delivered to an interior of the proximal hub 506. In some cases, the orientation of the fitting 512 relative to the proximal hub 506 may provide an indication of a direction of curvature for a curved distal region (not shown) of the elongate shaft 508. The elongate dilator 504 may be considered as representing the addition of an elongate dilator shaft 514 to the dilator hub 184 described with respect to FIG. 6. dilator hub 184.

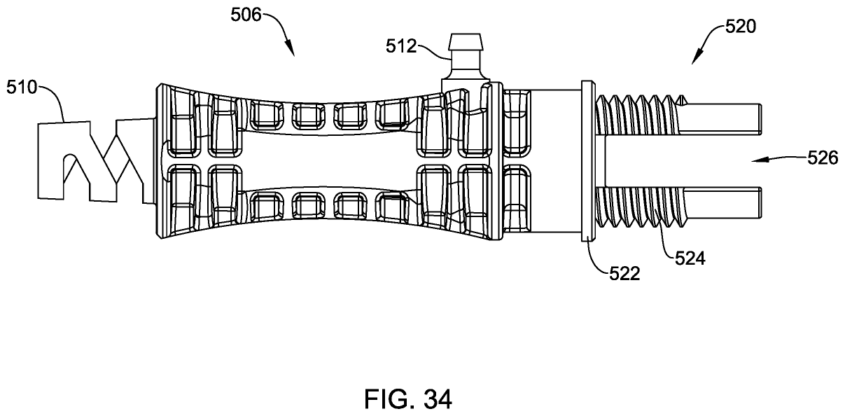

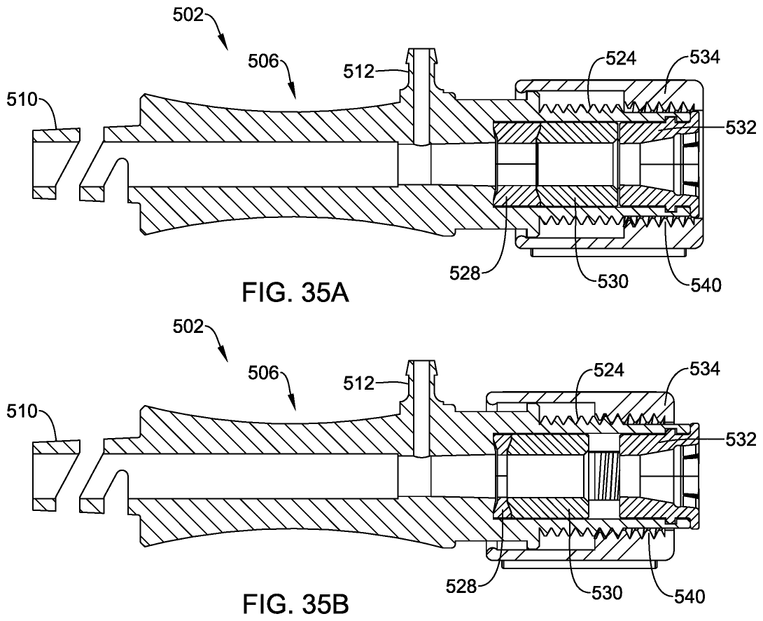

FIG. 34 is a side view of the proximal hub 506. The proximal hub 506 includes a proximal region 520. The proximal region 520 includes an annular flange 522, an outer threaded surface 524 and an elongate slot 526 that is cut into the proximal region 520. The elongate slot 526 is adapted to accommodate several additional components, as seen for example in FIGS. 35A and 35B. The additional components include a valve 528, a pusher 530, an inner member 532 and a valve nut 534.

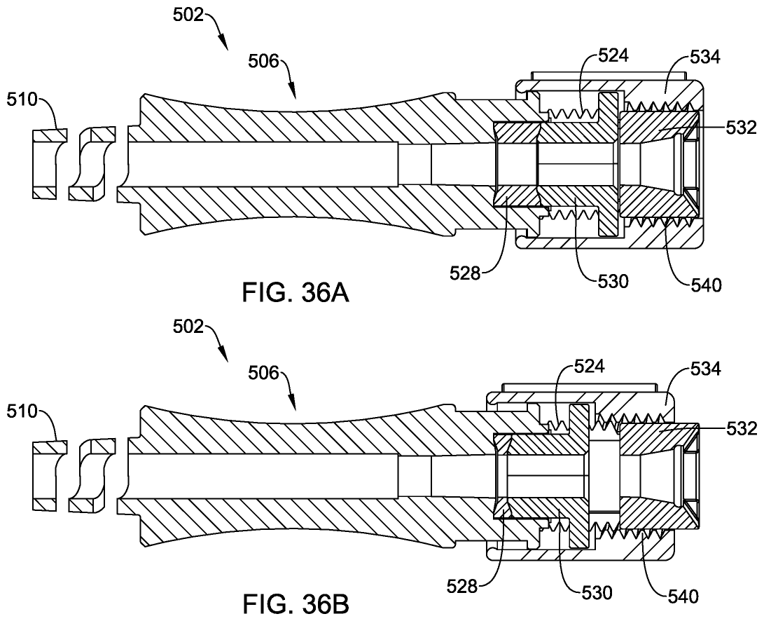

FIG. 35A is a schematic cross-sectional view showing the seal open, with free counterclockwise rotation and no thread engagement. As shown, the pusher 530 has not yet compressed the valve 528. The valve nut 534 includes an inner threaded surface 540 that engages the outer threaded surface 524 of the proximal hub 506. In FIG. 35B, the pusher 530 has moved distally and thus has compressed the valve 528. In comparing FIGS. 35A and 35B, it can be seen that the valve nut 534 has moved distally in causing the pusher 530 to contact and compress the valve 528. FIG. 36A is the same as FIG. 35A, but represents a cross-section taken 90 degrees rotated from the orientation in FIG. 35A. FIG. 36B is the same as FIG. 35B, but represents a cross-section taken 90 degrees rotated from the orientation in FIG. 35B. As can be seen, the internally threaded surface 540 only extends part of a length of the valve nut 534.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “superelastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, The devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device assembly, comprising:
a first elongate medical device comprising:
a first elongate shaft having a first proximal region and a first distal region; and
a first proximal hub secured to the first proximal region; and
a second elongate medical device comprising:
a second elongate shaft having a second proximal region and a second distal region; and
a second proximal hub secured to the second proximal region;
wherein the first elongate medical device is adapted to form a releasable axial and/or rotational lock with the second elongate medical device.

2. The medical device assembly of claim 1, wherein the second elongate shaft is adapted to fit within a lumen extending through the first elongate shaft.

3. The medical device of claim 2, wherein the second proximal hub is adapted to releasably couple to the first proximal hub when the second elongate shaft is disposed within the lumen extending through the first elongate shaft.

4. The medical device of claim 3, wherein the second proximal hub is adapted to form a releasable rotational lock with the first proximal hub.

5. The medical device of claim 3, wherein the second proximal hub is adapted to form a releasable axial lock with the first proximal hub.

6. The medical device assembly of claim 1, wherein the first elongate medical device is adapted to be advanced over a guidewire in order to reach an atrial septum.

7. The medical device assembly of claim 6, wherein the second elongate medical device is adapted to be advanced over the guidewire in combination with the first elongate medical device in order to create or enlarge an aperture formed in the atrial septum.

8. The medical device assembly of claim 1, wherein the first elongate medical device comprises a guide catheter.

9. The medical device assembly of claim 1, wherein the second elongate medical device comprises an elongate dilator.

10. The medical device assembly of claim 9, wherein the second proximal hub comprises a graspable profile.

11. A medical device assembly for accessing a left atrial appendage, comprising:
a guide catheter comprising:
a guide catheter shaft; and
a guide catheter hub secured to a proximal region of the guide catheter shaft; and
an elongate dilator comprising:
an elongate dilator shaft including a distal region adapted to create and/or enlarge an aperture in tissue; and
a dilator hub secured to a proximal region of the elongate dilator shaft;
wherein the guide catheter and the elongate dilator are adapted to form a releasable lock therebetween.

12. The medical device assembly of claim 11, wherein the elongate dilator shaft is adapted to fit within a lumen extending through the guide catheter shaft.

13. The medical device of claim 12, wherein the dilator hub is adapted to releasably couple to the guide catheter hub when the elongate dilator shaft is disposed within the lumen extending through the guide catheter shaft.

14. The medical device of claim 13, wherein the dilator hub is adapted to form a releasable rotational lock with the guide catheter hub.

15. The medical device of claim 13, wherein the dilator hub is adapted to form a releasable axial lock with the guide catheter hub.

16. The medical device assembly of claim 11, wherein the guide catheter is adapted to be advanced over a guidewire in order to reach an atrial septum.

17. The medical device assembly of claim 16, wherein the elongate dilator is adapted to be advanced over the guidewire in combination with the guide catheter in order to create or enlarge an aperture formed in the atrial septum.

18. The medical device assembly of claim 11, wherein the second proximal hub comprises a graspable profile.

19. A medical device assembly for implanting a left atrial appendage closure (LAAC) device, comprising:
a guide catheter comprising:
a guide catheter shaft; and
a guide catheter hub secured to a proximal region of the guide catheter shaft; and
an elongate dilator comprising:
an elongate dilator shaft including a distal region adapted to create and/or enlarge an aperture in tissue; and
a dilator hub secured to a proximal region of the elongate dilator shaft;
wherein the guide catheter and the elongate dilator are adapted to form a releasable lock therebetween.

20. The medical device assembly of claim 19, further comprising an LAAC device delivery catheter that is adapted to be advanced through the guide catheter once the elongate dilator has been removed.
